(19) United States
(12) Patent Application Publication
(10) Pub. No.: US 2009/0105260 A1
(43) Pub. Date: Apr. 23, 2009

(54) Molecules to Enhance Percutaneous Delivery and Methods for Discovery Therefor

(76) Inventors: Samir Mitragotri, Goleta, CA (US); Pankaj Karande, Troy, NY (US); Amit K. Jain, Goleta, CA (US)

Correspondence Address: BERLINER & ASSOCIATES, 555 WEST FIFTH STREET, 31ST FLOOR, LOS ANGELES, CA, 90013, US

(21) Appl. No.: 11/883,606

(22) PCT Filed: Jan. 17, 2006
(86) PCT No.: PCT/US2006/001964

§ 371 Date: Oct. 29, 2008

Related U.S. Application Data
(60) Provisional application No. 60/655,839, filed on Feb. 23, 2005.

(51) Int. Cl. A61K 31/53; C07D 251/26; C07C 43/30; C07D 211/06; A61K 31/445; A61K 31/03; G06F 19/00; G01N 21/25; A61K 31/495; A61K 31/075; C07D 241/04; C07C 17/00

(52) U.S. Cl. 514/241; 544/221; 568/591; 546/192; 544/403; 570/101; 514/715; 514/317; 514/252.12; 514/753; 356/326; 702/22

(57) ABSTRACT

An IR spectroscopic technique provides methods for measuring the irritation potential of a formulation and to assess the ability of molecules to enhance the permeability of substances into and through skin using samples comprising stratum corneum. Molecules are screened for their performance as chemical penetration enhancers using a unique in silico procedure that may be applied iteratively in an attempt to generate molecules showing successively higher performance. Both the irritation potential and the ability of the molecule to enhance penetration are considered in the in silico approach. The invention provides specific molecules that may be used in topical or transdermal formulations to improve the delivery of actives. The structures of compounds of the invention include: Formulas (I), (II), (III), (IV), (V), (IV) and analogs thereof.

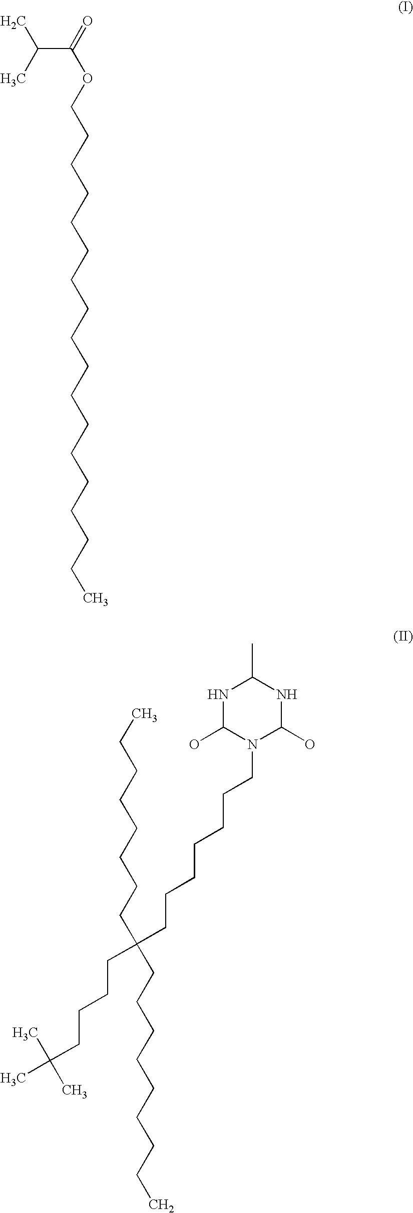

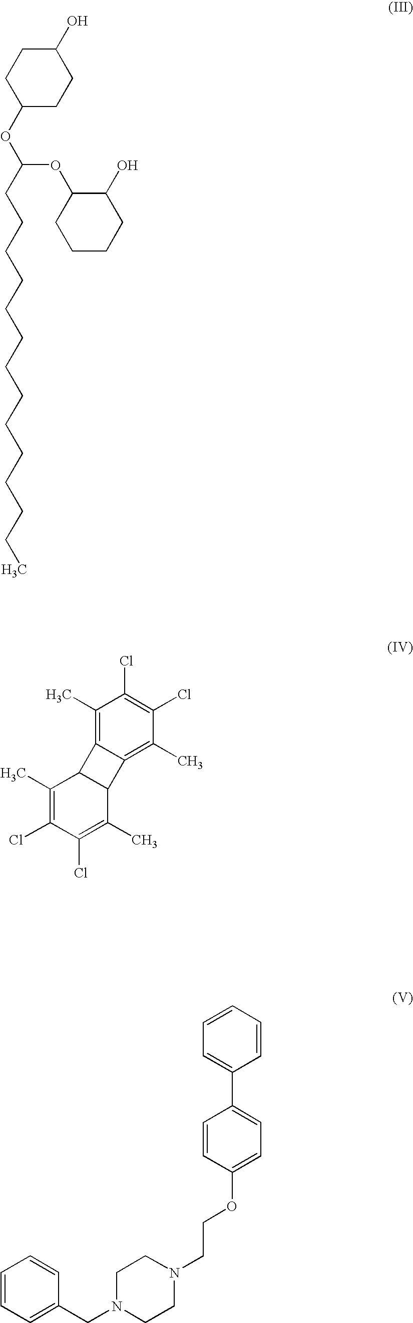

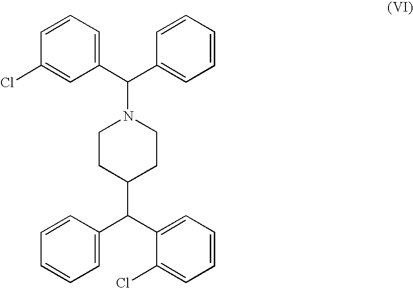

### FIELD

The invention includes molecules for the delivery of active ingredients such as drugs into and through skin and related screening methods.

### BACKGROUND

Skin permeation of exogenous molecules is of considerable interest for both pharmaceutical and cosmetic applications. Transdermal delivery provides an attractive approach for administration of drugs. Benefits of this non-invasive method of drug delivery over other modalities of administration may include (i) avoidance of first-pass liver metabolism, (ii) circumvention of exposure of the drug to the chemical rigors of the gastrointestinal tract, (iii) elimination of gastrointestinal distress, (iv) improvement of the safety and/or efficacy of drugs with short biological half-lives and/or narrow therapeutic windows, (v) reduction of adverse events, (vi) provision of a simple means for prompt interruption of dosing, and (vii) improvements in patient compliance. In the field of dermatology topical delivery of drugs is also often desirable, while in the area of cosmetics there is increasing interest in the delivery of skin care actives from topically applied formulations.

Human skin has evolved to impede the flux of exogenous molecules, making topical and transdermal delivery of actives difficult. In spite of the attractions of transdermal drug delivery only about a dozen drug molecules are at present available in this format in products approved by the Food and Drug Administration (FDA). It has been observed that delivery of molecules with molecular weights of more than 500 Da is particularly challenging. Bos (2000). To deliver effective amounts of actives across the skin, the natural transport barrier of the skin must often be compromised. The primary diffusion barrier of the skin is provided by the outermost layer of this organ, the stratum corneum (SC), a compact structure which includes corneocytes and lipid lamellae.

Several technological advances have been pursued in the past two decades to modify or circumvent the skin barrier including iontophoresis, sonophoresis and use of chemical penetration enhancers (CPEs). Prausnitz et al. (2004). CPEs are substances that act on the skin to reduce its diffusional resistance to the transport of therapeutics and other actives. CPEs may enhance the diffusion of molecules across skin by, for example, disrupting the corneocytes or the lipid bilayers of the stratum corneum. CPEs provide an attractive means for enhancing drug transport in the field of transdermal delivery. They allow design flexibility with formulation chemistry, are compatible with the possibility of patch application over a large skin area (>10 cm²) and provide the ability to deliver actives without the need of external physical delivery devices. Moreover, CPEs can also be incorporated into cosmetics and topical drug formulations to enhance the delivery of actives in those formats.

Several different classes of CPEs including surfactants, fatty acids and fatty esters have been studied for permeation enhancement and over 250 substances have been identified as chemical penetration enhancers. However, potent CPEs are often also potent irritants to the skin at the concentrations necessary to induce the desired level of penetration enhancement. They have thus been of limited practical use. Since the stratum corneum comprises non-viable, keratinized cells it is reasonable to suppose that disruption of its structure alone is not sufficient to induce irritation. However, CPEs are usually not selective towards the stratum corneum and eventually affect the viable cells of the epidermis thereby inducing irritation, for example, by interstitial release of cytokines and/or by triggering other inflammatory responses. Attempts have been made to synthesize novel CPEs, for example azone, to achieve therapeutic transport enhancement. However, achieving sufficient potency with CPEs with cosmetically and clinically acceptable irritancy has proved to be a challenging problem.

Discovery of new CPEs to increase skin permeability is highly desirable and, accordingly, this field has been an area of high activity in the last three decades. Santus et al. (1993); Asbill et al. (2000); Kanikkannan et al. (2000); Bauerova et al. (2001). However, the number of substances which have been identified to be chemical penetration enhancers is still very small when compared, for example, to the more than 25,000,000 organic and inorganic substances at present contained in the CAS registry (Chemical Abstracts Service, Columbus, Ohio, www.cas.org). The low number of substances that have been identified to be CPEs partly originates from difficulties in testing the ability of a molecule to enhance transport across this skin barrier without inducing unacceptable irritation, which at present is a slow and expensive process.

A traditional method of performing skin permeation studies, including of topical and transdermal drug delivery formulations as well as of ophthalmics, cosmetics, skin care products and pesticides, employs a vertical diffusion cell. Franz (1978). Permeation of a chemical agent from an upper donor well, through a skin sample, into a lower receptor well is assessed through analysis of the concentration of chemical agent in the donor and receptor wells, such as by high performance liquid chromatography. The diffusion cells introduced by Franz, and others, typically allow formulations to be tested one at a time and allow a single operator to test a few formulations per day. More recently, experimental methods and devices have been described which utilize miniaturized diffusion cells in array formats. U.S. Pat. No. 5,490,415; International Application Number PCT/US01/22167 published under International Publication Number WO 02/06518 A1; International Application Number PCT/US01/26473 published under International Publication Number WO 02/16941 A2; Karande et al. (2002). Devices utilizing arrays of diffusion cells may increase the rate at which formulations containing putative CPEs may be tested for their ability to enhance the delivery of actives. However, testing of very large numbers of materials to discover new CPEs is expensive and time consuming even with the use of parallel systems. Moreover, the use of such techniques is predicated on the ability to provide a supply of the material to be tested.

With respect to testing of irritancy of materials, the Draize rabbit skin test has served as a world standard for evaluating skin irritation and corrosion induced by chemicals for more than 50 years. Draize et al. (1944). Although different regulatory authorities have modified the procedure, this test basically measures the severity, speed of onset, and persistence/reversibility of skin reactions following the application of test samples to shaved rabbit skin under an occlusive or semi-occlusive dressing. However, such tests are slow and expensive and may cause pain and suffering to the animals used in the experiments.

Several in vitro alternatives to Draize test have therefore been developed that utilize (i) skin specimens or skin-equivalent organ cultures, (ii) keratinocyte, fibroblast and endothelial cell cultures or (iii) in vitro reconstituted biomolecules (liposomes and synthetic protein matrices). Osborn et al. (1994); Perkins et al. (1999); Newby et al. (2000); Faller et al. (2002). In all these in vitro tests, the substance in question is incubated with the test substrate. The test substrate is then analyzed using an approach such as measuring (i) histological integrity and electrical conductivity in case of skin, (ii) cell viability, cytokine release, growth, differentiation and metabolism in case of cell cultures, or (iii) permeability to fluorescence probes and turbidity of the matrix in case of synthetic substrates. Although current in vitro methods have evolved significantly over the last decade, they have failed to replace the traditional methods due to several practical and fundamental issues. Herzinger et al. (1995); Eun et al. (2000). Methods employing in vitro reconstituted biomolecules generally do not model the barrier properties of the stratum corneum, which is a significant limitation. Approaches utilizing skin specimens or cell cultures involve the use of biological samples, which usually have limited shelf lives and are difficult to handle. As a consequence, methods utilizing skin specimens or cell cultures can be expensive to apply in practice. In vitro approaches for measuring the irritation potential of molecules that avoid the use of biological samples while correctly accounting for the barrier properties of the stratum corneum would therefore have significant appeal. Also of great interest would be mathematical models of skin irritation that would allow predictions of the irritation potential of a molecule to be made without the need for physical experiments. Such mathematical models might in principle allow the exponential improvements in computational power that have been witnessed for over more than three decades to be leveraged to conveniently estimate the irritation potential of molecules at low cost.

Accordingly, it would be desirable to identify new molecules with low irritation potential that are effective at enhancing the transport of drugs across the skin barrier. Novel methods to accelerate the screening of the irritation potential of formulations and the ability of putative CPEs to enhance transport across skin are also desirable.

### SUMMARY

The inventions described and claimed herein have many attributes and embodiments including, but not limited to, those set forth or described or referenced in this Summary. The inventions described and claimed herein are not limited to or by the features or embodiments identified in this Summary, which is included for purposes of illustration only and not restriction.

The present invention provides, for example, methods for determining the irritation potential of a formulation using infrared (IR) spectroscopy. A substrate is employed, which may take the form of a skin sample or a sample consisting essentially of stratum corneum, and the formulation of interest applied to the substrate. The interaction of the formulation with the substrate causes changes to the infrared absorption spectrum of the stratum corneum to occur. These changes, which are evident in the Amide I band of the IR spectrum, for example, may be analyzed to quantify the irritation potential of the formulation. Measurement in the changes in the Amide I band may be facilitated by fitting the band to Gaussians. The IR spectrum may be measured, for example, using a Fourier transform infrared (FTIR) spectrometer at a resolution of about 2 cm⁻¹, or better. The method may be applied in a protocol where the spectrum is measured before and after application of formulation, in order that changes to the IR spectrum caused by the formulation may be more easily quantified and also may utilize solvent rinsing steps with, for example, deuterated solvents.

Other embodiments of the invention provide methods for discovering new chemical penetration enhancers in silico using molecular decriptors to compute performance attributes for sets of molecules. For example, in one embodiment solubility parameters and logP values are used to compute the ability of molecules in a set to enhance the transport of actives as well as the irritation potential of the molecule. The approach allows large compound collections to be screened for their performance as CPEs. In another embodiment the invention provides iterative approaches for attempting to improve the performance of molecules as CPEs by selecting the leading candidates at each iteration, modifying the leading candidates and screening the modified candidates in the following iteration for their performance as CPEs.

Other embodiments of the invention provide molecules such as

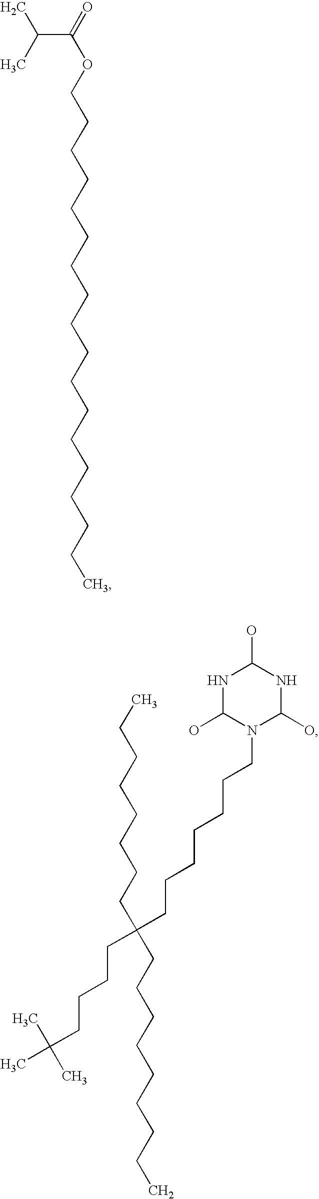

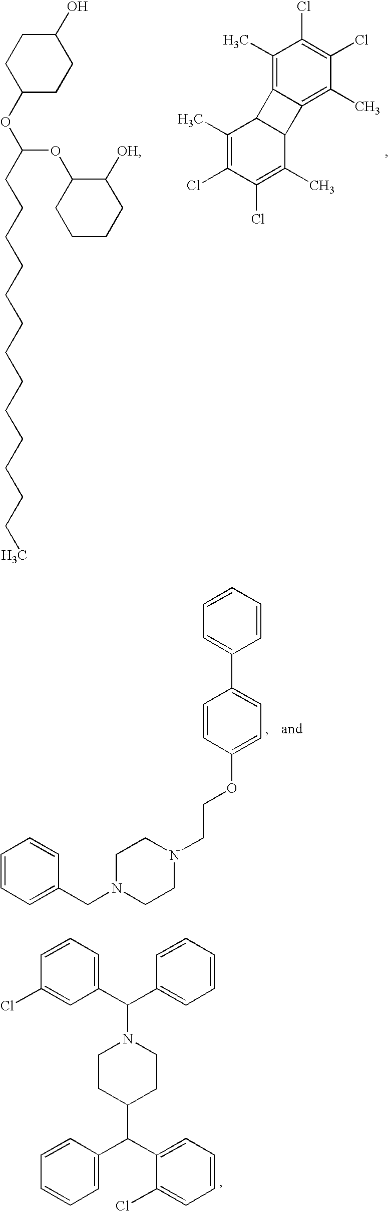

which may be used as CPEs. In one embodiment one or more of these molecules are incorporated into formulations and applied to the skin of a human or an animal for topical or transdermal delivery of actives.

### BRIEF DESCRIPTION OF THE DRAWINGS

FIG. 1 depicts the structure of skin and illustrates schematically the corneocytes of the stratum corneum embedded in a lipid bilayer matrix;

FIG. 2 depicts a portion of an FTIR spectrum of the stratum corneum;

FIG. 3 is a flow chart showing a sequence of steps useful for determination of the irritation potential of a formulation according to a preferred embodiment of the present invention;

FIG. 4 depicts the deconvolution of the Amide I band into Gaussians of a stratum corneum infrared spectrum before treatment with a formulation containing CPEs;

FIG. 5 depicts the deconcolution of the Amide I band into Gaussians of a stratum corneum infrared spectrum after treatment with a formulation containing 1.5% wt/vol lauric acid in a vehicle of 1:1 EtOD:D₂O;

FIG. 6 is a plot of change in integrated absorbance of the carbonyl stretching mode at 1650 cm⁻¹ of the stratum corneum against irritation potential measured with EpiDerm™produced by a series of formulations containing CPEs;

FIG. 7 is a plot of conductivity enhancement ratio against change in integrated absorbance of the symmetric methylene stretching modes, Δ(ν_{sym}CH₂), for stratum corneum samples treated with a variety of formulations containing CPEs;

FIG. 8 is a plot of Δ(ν_{sym}CH₂) against functions of molecular descriptors for formulations containing a variety of CPEs, showing separate correlations for CPEs associated with positive (close circles) and negative (open circles) values Δ(ν_{sym}CH₂);

FIG. 9 is a plot depicting a correlation between IP for formulations containing various CPEs and the ratio of hydrogen bonding forces (δₕ) to ratio of polar forces (δₚ) of the CPEs;

FIG. 10 is a flow chart showing a sequence of steps useful for in silico discovery of chemical penetration enhancers according to a preferred embodiment of the present invention;

FIG. 11 depicts extraction potential and fluidization potential for wild-type and mutant CPEs calculated from molecular descriptors;

FIG. 12 depicts molecular structures of some mutant fluidizers (I) stearyl methacrylate, (II) 1-(2-hydroxy-phenoxy), 1-(4-hydroxy-phenoxy) pentadecane, (III) 1-(8-octyl-8-(1,1-dimethylhexyl)heptadecane)-1,3,5-triazine-2,4,6-trione, (IV) 1-benzyl-4-(2-((1,1′-biphenyl)-4-yloxy)ethyl)piperazine, (V) 1,4-bis-((2-chloro-phenyl)-phenyl-methyl)-piperazine and (VI) 2,3,6,7-tetrakis(chloromethyl)-1,4,5,8-tetramethylbiphenylene;

FIG. 13 is a list of CPEs that may be characterized as anionic surfactants;

FIG. 14 is a list of CPEs that may be characterized as zwitterionic surfactants;

FIG. 15 is a list of CPEs that may be characterized as cationic surfactants;

FIG. 16 is a list of CPEs that may be characterized as nonionic surfactants;

FIG. 17 is a list of CPEs that may be characterized as fatty acids;

FIG. 18 is a list of CPEs that may be characterized as fatty esters;

FIG. 19 is a list of CPEs that may be characterized as sodium salts of fatty acids;

FIG. 20 is a list of CPEs that may be characterized as alkyl amines;

FIG. 21 is a list of CPEs that may be characterized as azone-like molecules;

FIG. 22 is a list of CPEs containing functional groups such as alcohols, ethers and carbonyl groups;

FIG. 23 is a list of CPEs whose interaction with samples of stratum corneum has been studied using IR spectroscopy;

FIG. 24 depicts a sample IR spectrum of the stratum corneum showing the symmetric methylene stretching mode (ν_{sym}CH₂) at 2850 cm⁻¹—the solid and dashed curves shows the absorbance of ν_{sym}CH₂ of a stratum corneum sample before and after treatment, respectively, with a formulation containing 1.5 wt/vol lauric acid in a vehicle of 1:1 EtOD:D₂O;

FIG. 25 is a plot of experimental ER/IP versus ER/IP predicted from molecular descriptors for CPEs associated with extractor behavior;

FIG. 26 is a plot of experimental ER/IP versus ER/IP predicted from molecular descriptors for CPEs associated with fluidizer behavior;

FIG. 27 is a bar graph showing best of category ER/IP values for 102 CPEs, which were each classified into one of ten categories;

FIG. 28 depicts the structure of limonene together with possible substitution points (labeled A and B) where functional groups may be added to limonene in an effort to develop CPEs showing improved performance;

FIG. 29 depicts examples of functional groups that may be substituted at point A on the limonene molecule in FIG. 28 in an effort to develop CPEs showing improved performance;

FIG. 30 depicts examples functional groups that may be substituted at point B on the limonene molecule in FIG. 28 in an effort to develop CPEs showing improved performance;

FIG. 31 depicts the pool size of mutant (solid line) and wild-type (dashed line) CPEs as a function of chemical descriptors that have been discovered to correlate with ER/IP; and

FIG. 32 is a bar chart depicting FP/IP_{Descriptor} for the mutant CPE stearyl methacrylate (SM) and a commonly used CPE in transdermal literature, oleic acid (OA), together with a comparison of inulin permeability enhancement achieved with formulations containing 1.5% wt/vol SM and OA.

### DETAILED DESCRIPTION

The following terms have the following meanings when used herein and in the appended claims. Terms not specifically defined herein have their art recognized meaning.

“Active component” or equivalently “active” means any substance that is known, or postulated, to provide a benefit when transported into or through skin and includes all such substances now known or later developed. Examples of active components include pharmaceuticals, vitamins, ultra violet (“UV”) radiation absorbers, cosmeceuticals, alternative medicines, skin care actives, and nutraceuticals. Active components can, by way of example but not limitation, be small molecules, proteins or peptides, genetic material, such as DNA or RNA, diagnostic or sensory compounds, agrochemicals, a component of a consumer product formulation, or a component of an industrial product formulation.

“Chemical penetration enhancer” or, equivalently, “penetration enhancer,” or “CPE” or “enhancer” means a substance used to modify, usually to increase, the rate of permeation through skin or other tissue of one or more substances in a formulation, and includes all such substances now known or later developed or discovered. See Santus et al. (1993). Various CPEs are listed below.

Surfactants: These are amphiphilic molecules with a hydrophilic head and a hydrophobic tail group. The tail length and the chemistry of the head group play an important role in determining their effect on skin permeability. Surfactants can be categorized into four groups, cationic, anionic, non-ionic, and zwitterionic depending on the charge on the head group. Prominent examples of surfactants that have been used for transdermal delivery include: Brij (various chain lengths), HCO-60 surfactant, Hydroxypolyethoxydodecane, Lauryl sarcosine, Nonionic surface active agents, Nonoxynol, Octoxynol, Phenylsulfonate, Pluronic, Polyoleates (nonionic surfactants), Rewopal HV10, Sodium laurate, Sodium oleate, Sorbitan dilaurate, Sorbitan dioleate, Sorbitan monolaurate, Sorbitan monooleates, Sorbitan trilaurate, Sorbitan trioleate, Span 20, Span 40, Span 85, Synperonic NP, Triton X-100, Tweens, Sodium alkyl sulfates, and alkyl ammonium halides.

Azone and related compounds: These compounds are also amphiphilic and possess a nitrogen molecule in their head group (preferably in the ring). The presence of a nitrogen atom in a ring creates a bulky polar head group with the potential for strong disruption of stratum corneum. Examples of such compounds include N-Acyl-hexahydro-2-oxo-1H-azepines, N-Alkyl-dihydro-1,4-oxazepine-5,7-diones, N-Alkylmorpholine-2,3-diones, N-Alkylmorpholine-3,5-diones, Azacycloalkane derivatives (-ketone, -thione), Azacycloalkenone derivatives, 1-[2-(Decylthio)ethyl]azacyclopentan-2-one (HPE-101), N-(2,2), Dihydroxyethyl dodecylamine, 1-Dodecanoylhexahydro-1-H-azepine, 1-Dodecyl azacycloheptan-2-one (azone or laurocapram), N-Dodecyl diethanolamine, N-Dodecyl-hexahydro-2-thio-1H-azepine, N-Dodecyl-N-(2-methoxyethyl)acetamide, N-Dodecyl-N-(2-methoxyethyl)isobutyramide, N-Dodecyl-piperidine-2-thione, N-Dodecyl-2-piperidinone, N-Dodecyl pyrrolidine-3,5-dione, N-Dodecyl pyrrolidine-2-thione, N-Dodecyl-2-pyrrolidone, 1-Farnesylazacycloheptan-2-one, 1-Farnesylazacyclopentan-2-one, 1-Geranyl azacycloheptan-2-one, 1, Geranylazacyclopentan-2-one, Hexahydro-2-oxo-azepine-1-acetic acid esters, N-(2, Hydroxyethyl)-2-pyrrolidone, 1-Laurylazacycloheptane, 2-(1-Nonyl)-1,3-dioxolane, 1-N-Octylazacyclopentan-2-one, N-(1-Oxododecyl)-hexahydro-1H-azepine, N-(1, Oxododecyl)-morpholines, 1-Oxohydrocarbyl-substituted azacyclohexanes, N-(1-Oxotetradecyl)-hexahydro-2-oxo-1H-azepine, and N-(1 Thiododecyl)-morpholines.

Solvents and related compounds: These molecules are solubility enhancers. Some of them also extract lipids, thereby increasing skin permeability. Examples of solvents include Acetamide and derivatives, Acetone, n-Alkanes (chain length between 7 and 16), Alkanols, diols, short-chain fatty acids, Cyclohexyl-1,1-dimethylethanol, Dimethyl acetamide, Dimethyl formamide, Ethanol, Ethanol/D-limonene combination, 2-Ethyl-1,3-hexanediol, Ethoxydiglycol (transcutol), Glycerol, Glycols, Lauryl chloride, Limonene, N-Methylformamide, 2-Phenylethanol, 3-Phenyl-1-propanol, 3-Phenyl-2-propen-1-ol, Polyethylene glycol, Polyoxyethylene sorbitan monoesters, Polypropylene glycol 425, Primary alcohols (tridecanol), Procter & Gamble system: small polar solvents (1,2-propane diol, butanediol, C3-6 triols or their mixtures and a polar lipid compound selected from C16 or C18 monounsaturated alcohol, C16 or C18 branched saturated alcohol and their mixtures), Span 20, Squalene, Triacetin, Trichloroethanol, Trifluoroethanol, Trimethylene glycol, Xylene, DMSO and related compounds.

Fatty alcohols, fatty acids, fatty esters, and related structures: These molecules are classic bilayer fluidizers. Examples of these enhancers include Aliphatic alcohols, Decanol, Lauryl alcohol (dodecanol), Linolenyl alcohol, Nerolidol, 1-Nonanol, n-Octanol, Oleyl alcohol, Butyl acetate, Cetyl lactate, Decyl N,N-dimethylamino acetate, Decyl N,N-dimethylamino isopropionate, Diethyleneglycol oleate, Diethyl sebacate, Diethyl succinate, Diisopropyl sebacate, Dodecyl N,N-dimethylamino acetate, Dodecyl (N,N-dimethylamino)-butyrate, Dodecyl N,N-dimethylamino isopropionate, Dodecyl 2-(dimethylamino)propionate, EO-5-oleyl ester, Ethyl acetate, Ethylaceto acetate, Ethyl propionate, Glycerol monoethers, Glycerol monolaurate, Glycerol monooleate, Glycerol monolinoleate, Isopropyl isostearate, Isopropyl linoleate, Isopropyl myristate, Isopropyl myristate/fatty acid monoglyceride combination, Isopropyl myristate/ethanol/L-lactic acid (87:10:3) combination, Isopropyl palmitate, Methyl acetate, Methyl caprate, Methyl laurate, Methyl propionate, Methyl valerate, 1-Monocaproyl glycerol, Monoglycerides (medium chain length), Nicotinic esters (benzyl), Octyl acetate, Octyl N,N-dimethylamino acetate, Oleyl oleate, n-Pentyl N-acetylprolinate, Propylene glycol monolaurate, Sorbitan dilaurate, Sorbitan dioleate, Sorbitan monolaurate, Sorbitan monooleates, Sorbitan trilaurate, Sorbitan trioleate, Sucrose coconut fatty ester mixtures, Sucrose monolaurate, Sucrose monooleate, Tetradecyl N,N-dimethylamino acetate, Alkanoic acids, Capric acid, Diacid, Ethyloctadecanoic acid, Hexanoic acid, Lactic acid, Lauric acid, Linoelaidic acid, Linoleic acid, Linolenic acid, Neodecanoic acid, Oleic acid, Palmitic acid, Pelargonic acid, Propionic acid, Vaccenic acid, α-Monoglyceryl ether, EO-2-oleyl ether, EO-5-oleyl ether, EO-10-oleyl ether, Ether derivatives of polyglycerols and alcohols (1-O-dodecyl-3-O-methyl-2-O-(29, 39-dihydroxypropyl)glycerol), L-α-amino-acids, Lecithin, Phospholipids, Saponin/phospholipids, Sodium deoxycholate, Sodium taurocholate, and Sodium tauroglycocholate.

Others: Aliphatic thiols, Alkyl N,N-dialkyl-substituted amino acetates, Anise oil, Anticholinergic agent pretreatment, Ascaridole, Biphasic group derivatives, Bisabolol, Cardamom oil, 1-Carvone, Chenopodium (70% ascaridole), Chenopodium oil, 1,8 Cineole (eucalyptol), Cod liver oil (fatty acid extract), 4-Decyloxazolidin-2-one, Dicyclohexylmethylamine oxide, Diethyl hexadecylphosphonate, Diethyl hexadecylphosphoramidate, N,N-Dimethyl dodecylamine-N-oxide, 4,4-Dimethyl-2-undecyl-2-oxazoline, N-Dodecanoyl-L-amino acid methyl esters, 1,3-Dioxacycloalkanes, (SEPAs), Dithiothreitol, Eucalyptol (cineole), Eucalyptus oil, Eugenol, Herbal extracts, Lactam N-acetic acid esters, N-Hydroxyethalaceamide, 2-Hydroxy-3-oleoyloxy-1-pyroglutamyloxypropane, Menthol, Menthone, Morpholine derivatives, N-Oxide, Nerolidol, Octyl-β-D-(thio)glucopyranosides, Oxazolidinones, piperazine derivatives, Polar lipids, Polydimethylsiloxanes, Poly[2-(methylsulfinyl)ethyl acrylate], Polyrotaxanes, Polyvinylbenzyldimethylalkylammonium chloride, Poly(N-vinyl-N-methyl acetamide), Prodrugs, Saline, Sodium pyroglutaminate, Terpenes and azacyclo ring compounds, Vitamin E (α-tocopherol), Ylang-ylang oil, N-Cyclohexyl-2-pyrrolidone, 1-Butyl-3-dodecyl-2-pyrrolidone, 1,3-Dimethyl-2-imidazolikinone, 1,5 Dimethyl-2-pyrrolidone, 4,4-Dimethyl-2-undecyl-2-oxazoline, 1-Ethyl-2-pyrrolidone, 1-Hexyl-4-methyloxycarbonyl-2-pyrrolidone, 1-Hexyl-2-pyrrolidone, 1-(2 Hydroxyethyl)pyrrolidinone, 3-Hydroxy-N-methyl-2-pyrrolidinone, 1-Isopropyl-2-undecyl-2-imidazoline, 1-Lauryl-4-methyloxycarbonyl-2-pyrrolidone, N-Methyl-2-pyrrolidone, Poly(N-vinylpyrrolidone), Pyroglutamic acid esters, Acid phosphatase, Calonase, Orgelase, Papain, Phospholipase A-2, Phospholipase C and Triacylglycerol hydrolase.

“Descriptor” means a quantity associated with a molecular entity. Examples of descriptors include, but are not limited to, molecular charge, dipole and higher order moments of the molecular charge density, molecular weight, molecular volume, molecular surface area, number of rotatable bonds, partition coefficients (e.g. water-octanol partition coefficient), density, melting point, boiling point, cohesive energy density, solubility parameters and solubilities;

“Irritation potential” means a quantitative measure of the degree of irritation that a composition produces when applied to skin. Irritation potential may be measured in vivo using animals or humans. For example, in vivo irritation potential in humans may be measured by the 21-day cumulative irritation test. Berger (1982). Irritation potential may also be measured in vitro. In one approach to measurement of irritation potential, reconstructed human epidermis equivalents may be employed such as EpiDerm™ or EPISKIN™. Faller et al. (2002);

“Formulation” means a single substance or a mixture of more than one substance. A formulation may, for example, contain one active component and multiple excipients. Formulations can take many forms, which include, without limitation, solids, semisolids, liquids, solutions, emulsions, suspensions, triturates, gels, films, foams, pastes, ointments, adhesives, highly viscoelastic liquids and any of the foregoing having solid particulates dispersed therein.

“Sample” a small part of something intended as representative of the whole;

“Skin” means the tissue layer forming the external covering of the body of a human or an animal, which is in turn characterized by a number of sub-layers such as the dermis, the epidermis and the stratum corneum. Skin also means skin-equivalent organ cultures such as EpiDerm™ or EPISKIN™; and

“Pharmaceutical” or, used interchangeably, “drug” means any substance or compound that has a therapeutic, disease preventive, diagnostic, or prophylactic effect when administered to an animal or a human. The term pharmaceutical includes prescription drugs and over the counter drugs. The molecular structures of drugs can often be characterized as small molecules, peptides, proteins and antibodies although other structures also include, for example, oligonucleotides and polysaccharides. Examples of pharmaceuticals include, but are not limited to, drugs of the following types: adrenergic agent; adrenocortical steroid; adrenocortical suppressant; aldosterone antagonist; amino acid; anabolic; analeptic; analgesic; anesthetic; anorectic; anti-acne agent; anti-adrenergic; anti-allergic; anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; antiulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid suppressant; gastrointestinal motility effector; glucocorticoid; hair growth stimulant; hemostatic; histamine H2 receptor antagonists; hormone; hypocholesterolemic; hypoglycemic, hypolipidemic; hypotensive; imaging agent; immunizing agent; immunomodulator; immunoregulator; immunostimulant; immunosuppressant, keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine A1 antagonist; serotonin antagonist; serotonin inhibitor; serotonin receptor antagonist; steroid; thyroid hormone; thyroid inhibitor; thyromimetic, tranquilizer; amyotrophic lateral sclerosis agent; cerebral ischemia agent; Paget's disease agent; unstable angina agent; vasoconstrictor; vasodilator; wound healing agent, xanthine oxidase inhibitor.

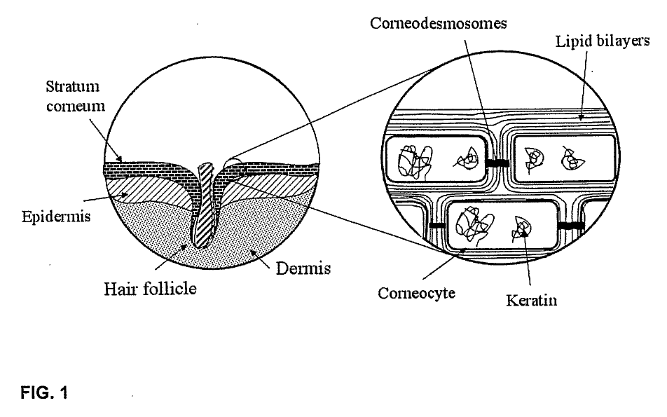

Transdermal drug delivery is an attractive method for systemic administration of actives and can be used to circumvent first pass metabolism and provide a sustained drug release for a prolonged period of time. Topical delivery allows a drug to be applied directly to the area of skin to be treated, which can be useful, for example, in the field of dermatology to localize the pharmaceutical to the site in the body where treatment is necessary and to minimize side effects. However, skin has evolved to impede the flux of toxins into the body and consequently offers a very low permeability to the movement of most foreign molecules. FIG. 1 illustrates schematically the structure of skin. The stratum corneum, the outermost layer of the skin, is primarily responsible for the skin's diffusion barrier. It possesses a unique hierarchical structure of a lipid rich matrix with embedded corneocytes and is typically about 15 μm in thickness. Bouwstra (1997). Corneocytes are non viable cells. These terminally differentiated keratinocytes possessing a proteinaceous core surrounded by a relatively impermeable cornified lipid envelope. Overcoming the skin barrier safely and reversibly is a fundamental problem that persists today in the field of dermal delivery of actives. Although more than two hundred and fifty chemical enhancers including surfactants, azone and related chemicals, fatty acids, fatty alcohols, fatty esters, and organic solvents have been tested to increase transdermal drug transport, only a handful are actually used in practice. Berti et al. (1995). This discrepancy results from the fact that among all the enhancers that have been considered, only a few induce a significant (therapeutic) enhancement of drug transport with acceptable levels of irritation. Walters (1989); Finnin (1999). It is highly desirable to find CPEs which show improved performance and that are effective at enhancing penetration of molecules across the skin while minimizing the irritation response. The number of molecules which have been identified to be CPEs represents a very small fraction of substances known in the chemical literature, a reflection of the fact that screening of molecules for their performance as CPEs is a slow and cumbersome undertaking. The present invention provides new CPEs and new methods for discovering CPEs.

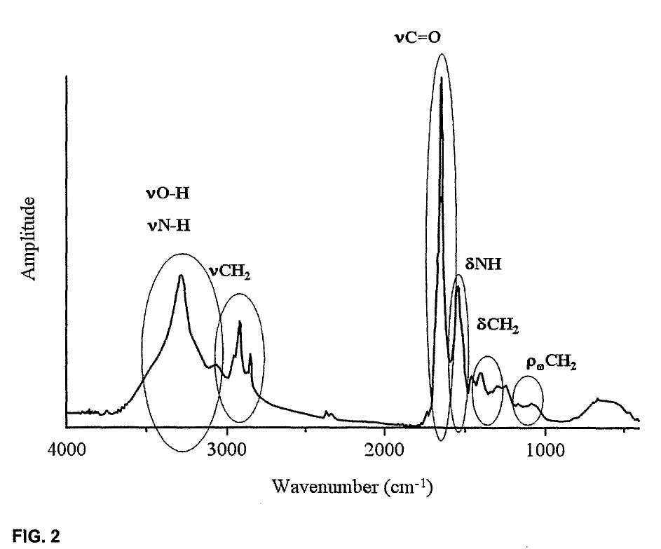

One embodiment of the present invention provides methods for measuring the irritation potential of a formulation using infrared spectroscopy. IR spectroscopy is a method that has found wide use in many branches of chemistry and that can be used to obtain information about the vibrational modes of molecules. IR spectra may be collected on equipment such as Fourier transform infrared spectrometers and dispersive infrared spectrometers. Bellamy (1958); Painter et al. (1982); Mendelsohn (1986). FIG. 2 shows the IR spectrum of a sample of stratum corneum measured with a Fourier transform infrared spectrometer. A series of peaks may be seen in the spectrum that arise from the vibrational modes of the molecules in the SC. Prominent peaks in the spectrum include the symmetric and anti-symmetric vibration modes of CH₂ functions (νCH₂ peaks at ˜2850 cm⁻¹ and ˜2920 cm⁻¹), the Amide I peak which arises from the stretching mode of C═O groups (νC═O at ˜1650 cm⁻¹), the Amide II peak which arises from NH in-plane bending and CN stretching (δNH at ˜1560 cm⁻¹) and features arising from wagging modes of CH₂ (ρ_{ω}CH₂ from ˜1360 cm⁻¹ to ˜1180 cm⁻¹). Both the lipids and proteins in the stratum corneum contribute to the IR spectrum. IR spectroscopy on biological samples is an established technique for inferring information on the secondary structures of proteins. See Jabs.

IR spectroscopy is a technique which has been previously utilized in the study of skin to obtain information about interactions of the stratum corneum with a variety of chemicals. Kai et al. describe experiments in which attenuated total reflectance FTIR spectra were measured on samples of murine skin which had been pretreated with alcohols such as ethanol, butanol, hexanol and octanol. Kai et al. (1989). Kai et al. concluded on the basis of their ETIR data that the major action of ethanol following the application procedure described in the paper is lipid extraction. Subsequently, Bommannan et al. performed attenuated total reflectance FTIR spectroscopy to determine the effects of ethanol on human stratum corneum in vivo. Bommannan et al. (1991). Bommannan et al. utilized FTIR data to conclude that a thirty minute treatment of the skin with pure ethanol (i) induced a transient decrease in the intensity and frequency of the C—H asymmetric stretching vibration (which originate from acyl chains of the intercellular lipid domains of the stratum corneum) (ii) cause observable increases in the spectral absorbances associated with ethanol and (iii) extracted appreciable amounts of the lipid from the stratum corneum. More recently Goates and Knutson used FTIR to investigate the influence of alcohol chain length on polar compound permeation in human skin. Goates et al. (1994). Goates and Knutson measured changes in the center of gravity and band width of the Amide I band of FTIR spectra of skin samples which had been treated with solutions containing methanol, ethanol and 2-propanol. The authors concluded that removal of stratum corneum proteins and lipid components appeared to be the primary source of alcohol-enhanced permeation of polar solutes through skin. These previous studies have provided qualitative information about the mechanisms of action of CPEs on the stratum corneum.

Surprisingly, it has been discovered that IR spectroscopy of the stratum corneum can be used as a method to obtain quantitative information about the irritation potential of a formulation. Irritation is generally believed to be caused by interactions of constituents of a formulation with the viable cells of the epidermis resulting in interstitial release of cytokines and/or triggering of other inflammatory responses. It is therefore unexpected that IR spectra, providing information about the vibrational modes of the molecules in the non-viable stratum corneum with its unique lipid matrix structure, should provide an approach for obtaining quantitative information on the irritation potential of a formulation.

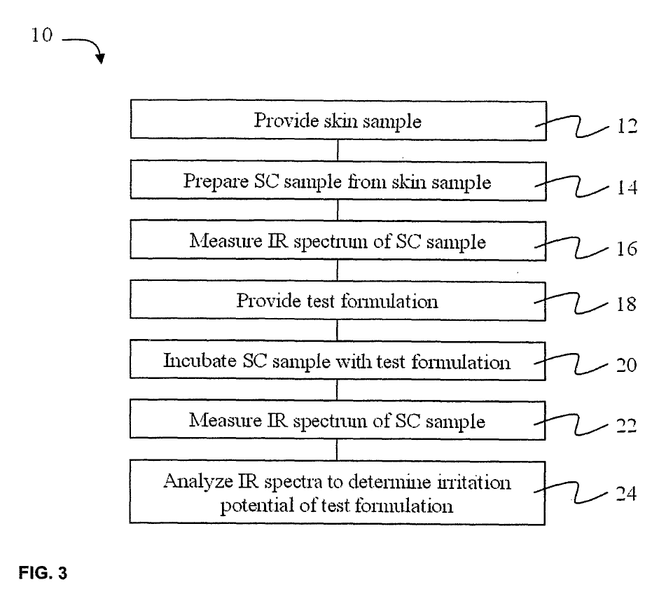

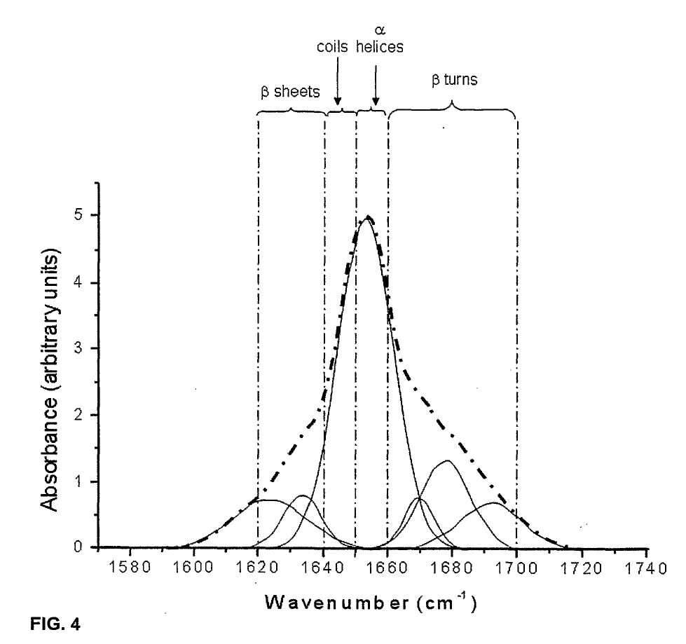

FIG. 3 is a flow diagram showing a sequence of steps that may be applied to determine the irritation potential of a formulation using IR spectroscopy according to a preferred embodiment of the invention. At the first step, 12, a sample of skin is provided. In preferred embodiments of the invention the skin is ex vivo porcine, murine or human skin. Next, at step 14, the stratum corneum is removed from the skin sample. Any suitable method may be employed for this purpose including treatment of the skin with chemicals (e.g. 2M sodium bromide). In a preferred embodiment of the invention this is accomplished by heat stripping following the approach described, for example, by Kligman et al. Kligman et al. (1963). The resulting membrane may be cut to a size suitable for use in an IR spectrometer. Preferred areas of the SC sample are slightly larger than the area addressed by the beam in the IR spectrometer employed. In a preferred embodiment of the invention this area may measure between about 0.5 cm² and 5 cm². At step 16 an IR spectrum of the stratum corneum is measured prior to treatment with the formulation. In a preferred embodiment of the invention a stratum-corneum drying period of at least 48 hours is allowed between removing the stratum corneum from the skin and measuring the IR spectrum. The stratum corneum may, for example, be dried at ambient temperature and pressure. It is preferred that the IR spectrum is measured as a transmission spectrum utilizing a suitable sample holder to hold the sample in the beam of the instrument. In a preferred embodiment of the invention the spectra is measured using a Fourier transform infra red spectrometer with a resolution of 2 cm⁻¹ or better. In a preferred embodiment of the invention several spectra are measured and the resulting traces averaged to reduce measurement noise. It is preferred that sufficient numbers of scans are performed in order to ensure that in the final measurement noise to signal ratio is less than about 0.3.

At step 18 a test formulation of interest is provided. In a preferred embodiment of the invention the test formulation may take the form of a solution of a chemical penetration enhancer or putative chemical penetration enhancer dissolved in any suitable solvent, or mixture of solvents. In a preferred embodiment of the invention the molecules in the formulation are deuterated such that OH groups are replaced with OD groups. At step 20 the SC samples are incubated with the test formulation. The formulation may be applied to one or both surfaces of the stratum corneum. In a preferred embodiment of the invention the SC samples are completely immersed in the test formulation. The formulation may be held in any suitable container for this purpose, for example screw-top vials. It is preferred that the incubation period is between 1 and 48 hours in duration and conducted at a temperature between 20° C. and 40° C. At the conclusion of the incubation period, in a preferred embodiment of the invention, the SC sample is thoroughly rinsed with a solvent or mixture of solvents to remove any excess test formulation from the stratum corneum. It is preferred that the solvents used for this purpose are deuterated such that OH groups in the formulation are replaced with OD groups. Any suitable solvents may be used for this purpose. In preferred embodiments of the invention EtOD or D₂O or a mixture thereof are used to remove excess test formulation from the stratum corneum. After removing the test formulation it is preferred that the sample of stratum corneum is allowed to dry for at least 48 hours before proceeding to the next step.

At step 22 a second IR spectrum is collected from the sample. It is preferred that the IR spectrum is measured using a Fourier transform infra red spectrometer with a resolution of 2 cm⁻¹ or better, in a transmission geometry. In a preferred embodiment of the invention several spectra are measured and the resulting traces averaged to reduce measurement noise. It is preferred that sufficient numbers of scans are performed in order to ensure that in the final measurement noise to signal ratio is less than about 0.3. In a preferred embodiment of the invention the IR spectrum is converted into an absorbance spectrum before analysis. This can typically be accomplished utilizing software provided by the manufacturers of IR spectrometers.

At step 24 the IR spectra are analyzed in order to determine the irritation potential of the formulation. In a preferred embodiment of the invention the regions of the before and after spectra corresponding to the Amide I band are analyzed. The Amide I band is generally the most intense band in protein IR spectra and it is sensitive to the protein conformation. This band can be deconvoluted to obtain contributions that may be interpreted to arise from the four broad secondary protein structures: β sheets (1640-1620 cm⁻¹), random coils (1650-1640 cm⁻¹), α helices (1660-1650 cm⁻¹) and anti-parallel β sheets and β turns (1695-1660 cm⁻¹).

In a preferred embodiment of the invention the spectra are smoothed, base line corrected and converted to CSV format for further processing. These steps may be accomplished using software provided by manufacturers of IR spectrometers. For example, the OMNIC™ software (Thermo Electron Corporation, Waltham, Mass. www.thermo.com) may be used for this purpose. The Amide I band may then be decomposed into a number of Gaussians, for example, by exporting the smoothed sprectra to the Origin software (OriginLab Corporation, Northampton, Mass., www.originlab.com) and deconvoluting the spectra using methods described in Byler. Byler et al. (1986). In a preferred embodiment of the invention the number of Gaussians used to fit the peak is sufficient to yield a χ² of 0.999 or better. In a preferred embodiment of the invention the integrated absorbance in the region 1660-1650 cm⁻¹ is measured by fitting the Amide I band to a number of Gaussians and computing the sum of the areas under the Gaussians whose maxima lie in the range 1660-1650 cm⁻¹. Once the Amide I band is deconvoluted into Gaussians a quantity Δ(νC═O) may be calculated by taking the difference between the integrated absorbance in the region between 1660-1650 cm⁻¹ before and after treatment with formulation.

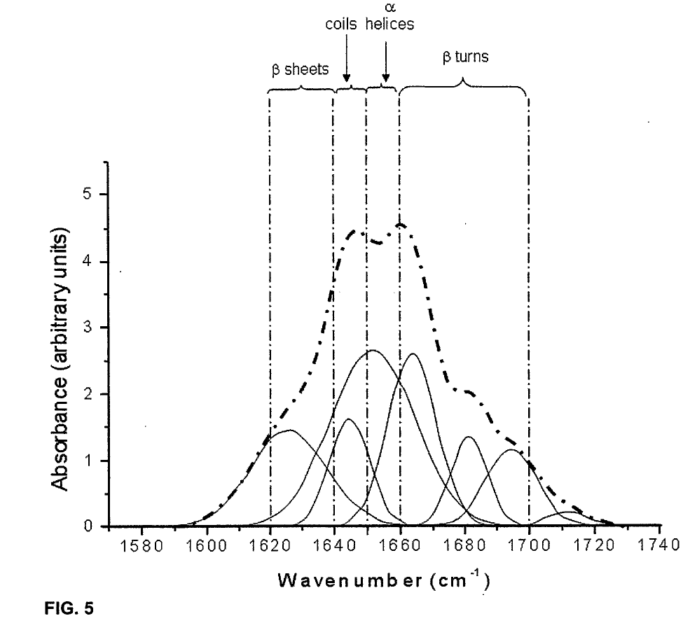

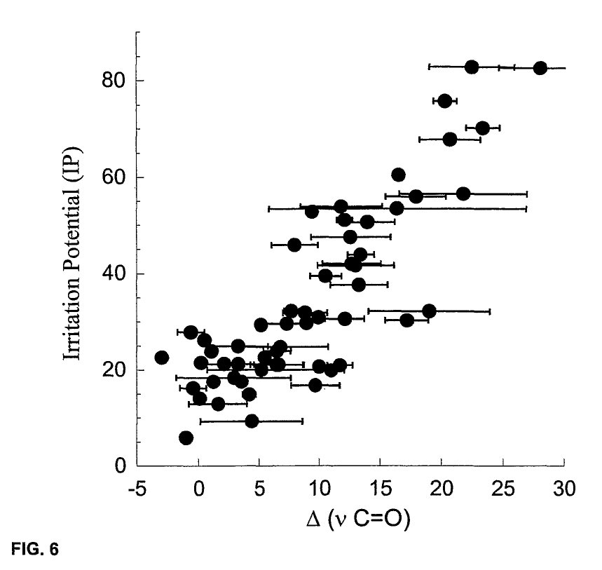

FIG. 4 and FIG. 5 provide an examples of Amide I bands of IR spectra of the stratum corneum measured before and after treatment with a formulation containing 1.5% wt/vol of the CPE lauric acid. FIG. 6 is a plot of Δ(νC═O) versus irritation potential measured using EpiDerm™, a skin equivalent organ culture (MatTek Corporation, Ashland, Mass. www.mattek.com), for a series of formulations containing different CPEs. A strong correlation between Δ(νC═O) and the EpiDerm™-measured irritation potential is evident. Further information relating to FIG. 4, FIG. 5 and FIG. 6 may be found by reference to Example 1 below.

Skin samples typically show significant sample-to-sample variability and in a preferred embodiment of the invention measurements of the change in the infrared spectra are measured on several skin samples and the results averaged.

It will be appreciated that the utilization of SC as a test substrate is advantageous compared to epidermis or full thickness skin since it can be lyophilized and easily handled. In contrast to measurements of irritation potential using cell culture based approaches, no sterile or other special cell culture procedures are required. In this sense, the stratum corneum can be handled as a material as opposed to a biological substance.

While FIG. 3 provides a sequence of steps that may be applied to determine the irritation potential of a formulation according to a preferred embodiment of the invention, numerous variations of the approach may be applied while remaining within the scope of the present invention. For example, the use of a stratum corneum sample that has been removed from the epidermis is not a requirement of the method. IR spectra may, for example, be collected either in vitro or even in vivo on samples where the stratum corneum is in contact with the epidermis using attenuated total reflectance infrared spectroscopy. Takashi et al. (1990); Bommannan et al. (1991). Similarly collection of IR spectra before and after incubation of the stratum corneum sample is not a requirement and analysis may be confined to post-treated skin samples.

The present invention may be beneficially applied in many ways and is generally applicable to compositions that may be applied to skin. For example it may be used to study the irritation potential of formulations containing CPEs or putative CPEs. It may be used in the development or testing of formulations for cosmetics, transdermal drug delivery applications, topical drug delivery applications as well as adhesives for patches, medical devices and medical dressings.

In addition it will be recognized that the present invention may be used to screen for topical and transdermal drug formulations showing low irritation potential. Many actives such as personal care actives, drugs and other materials that are used in topical and transdermal drug delivery formulations are skin irritants. Examples of actives that may cause skin irritation include, but are not limited to, certain sunscreens, hydroxy acids, in particular x-hydroxy acids (glycolic, lactic, malic, citric, tartaric, mandelic, etc.) and β-hydroxy acids, especially salicylic acid and its derivatives, keto acids, in particular in α- and β-form, derivatives of hydroxy or keto acids, especially in α- and β-form, retinoids (retinol and its esters, retinal, retinoic acid and its derivatives), anthralins (dioxyanthranol), anthranoids, peroxides (in particular benzoyl peroxide), minoxidil and its derivatives, lithium salts, antiproliferating agents such as 5-fluorouracil or methotrexate, certain vitamins such as vitamin D and its derivatives and vitamin B9 and its derivatives, hair tints or dyes (para-phenylenediamine and its derivatives, aminophenols), perfuming alcoholic solutions (fragrances, eaux de toilette, aftershave and deodorants), antiperspirants (some aluminum salts); hair-removing or permanent-waving active agents (thiols), depigmenting agents (hydroquinone), capsaicin, antilouse active agents (pyrethrin), ionic and nonionic detergent agents and propigmenting agents (dihydroxyacetone, psoralens and methylangecilins), and mixtures thereof. It will be appreciated that the irritation potential of a formulation is dependent on its composition. For example, US Patent Application 2003/0124202 A1 is directed to the use of the divalent cation strontium as an ingredient to provide fast-acting, efficient and safe topical skin anti-irritant effects. The present invention may be beneficially applied to develop compositions showing reduced irritation potential and to search for ingredients and ingredient combinations that minimize irritation potential.

Computational methods for the design and optimization of molecules have had a significant impact on the life sciences industry. In silico methods are widely used by pharmaceutical and biotechnology companies in the fields of genomics, target identification and validation, lead discovery and optimization, and drug development. A number of companies provide capabilities such as simulation, bioinformatics and cheminformatics software in this area including, for example, Accelrys (San Diego, Calif. www.accelrvs.com), Advanced Chemistry Development (Toronto, Ontario, Canada www.acdlabs.com), MDL (San Leandro, Calif. www.mdli.com) and Tripos (St. Louis, Mo. www.tripos.com). While simulation methods are well established and widely used, general approaches for determining the performance of molecules as chemical penetration enhancers for dermal delivery of actives have been unavailable. Kanikkannan et al. have reviewed some structure activity relationships that have been proposed in the field of chemical penetration enhancers. Kanikkannan et al. (2000). However, the structure activity relationships discussed by Kanikkannan et al. do not provide a general framework for quantitative comparison of key performance attributes of chemical penetration enhancers.

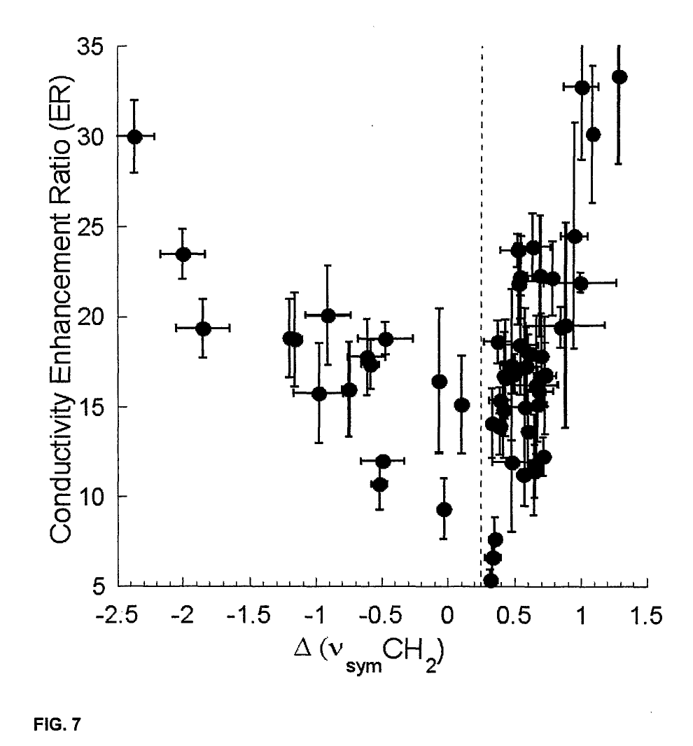

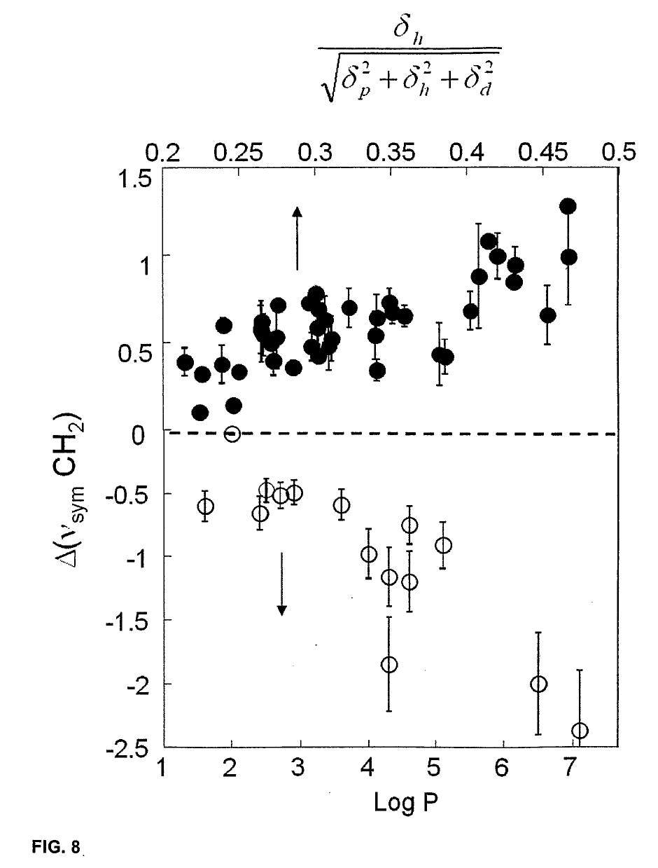

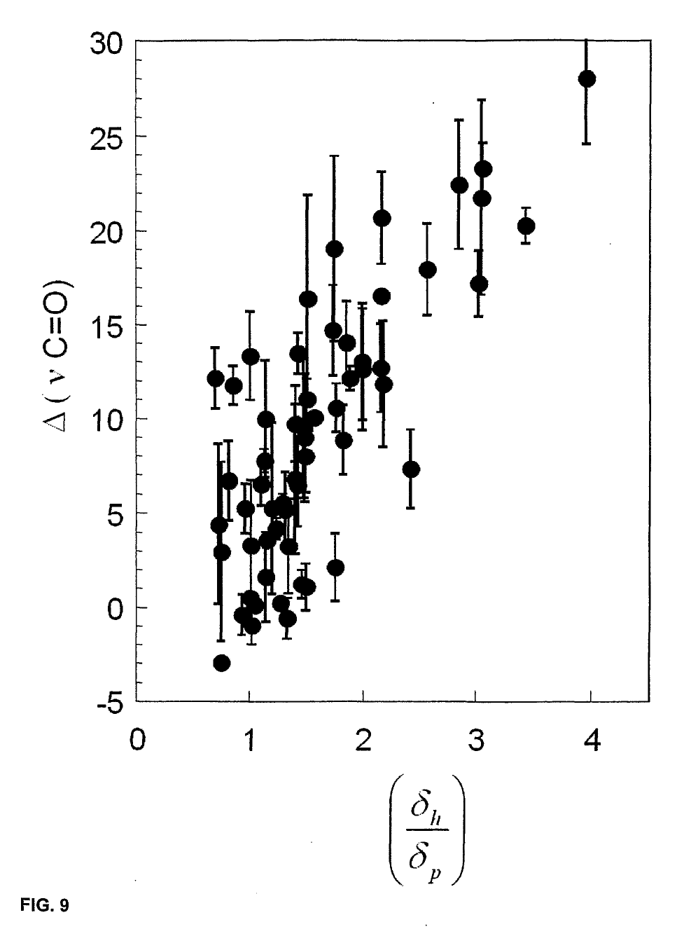

It has been discovered that quantitative information about two key elements of CPE performance, namely, the ability of a molecule to enhance the permeability of actives into or through skin and the irritation potential of a formulation containing the molecule, can be developed from descriptor information. Conductivity enhancement ratios (ER) may be used to measure the ability of formulations containing CPEs to enhance the transport of actives across skin. Karande et al. (2002). It has been discovered that the ER value of a formulation shows a correlation with changes in features of IR spectra measured before and after treatment with the formulation. FIG. 7 depicts experimentally measured conductivity enhancement ratio (ER) for formulations containing a series of CPEs against change in the area of the symmetric methylene stretching peak, Δ(ν_{sym}CH₂), measured with IR spectroscopy of stratum corneum samples before and after incubation with formulations containing the CPEs. It may be seen that Δ(ν_{sym}CH₂) shows two separate linear correlations with ER depending on whether Δ(ν_{sym}CH₂) is positive or negative. It has also been discovered that changes in Δ(ν_{sym}CH₂) for formulations containing CPEs may be correlated with molecular descriptors in particular solubility parameters (when Δ(ν_{sym}CH₂) positive) and logP (when Δ(ν_{sym}CH₂) negative), as depicted in FIG. 8. Moreover, Δ(νC═O), which as previously discussed correlates with the irritation potential of a formulation, may also be correlated with solubility parameters of the CPE as depicted in FIG. 9. Consequently, two key attributes of CPEs may be examined by calculation of molecular desciptors. This leads to a novel and rapid method of screening compound collections for the performance of members as CPEs and to approaches for in silico design and optimization of CPE molecules.

Without being bound by theory, a reduction in the integrated absorbance of the methylene stretching modes on treatment of the stratum corneum with a formulation, indicated by a positive Δ(ν_{sym}CH₂), may be interpreted to indicate a decrease in the lipid content or lipid extraction from the bilayers of the stratum corneum. Conversely, an increase in the peak area, or a negative value of Δ(ν_{sym}CH₂), may be interpreted to indicate partitioning of CPE molecules into the lipid bilayers of the stratum corneum. CPEs which cause increases and reductions in Δ(ν_{sym}CH₂) may be termed extractors and fluidizers, respectively. It is a surprising and unexpected result that, regardless of their chemical nature, the performance of extractor and fluidizer CPEs, as measured by ER values, show separate linear correlations with Δ(-ν_{sym}CH₂).

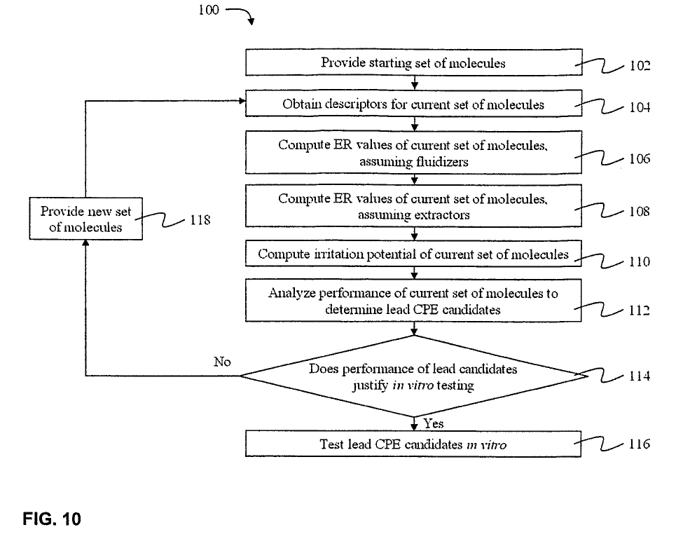

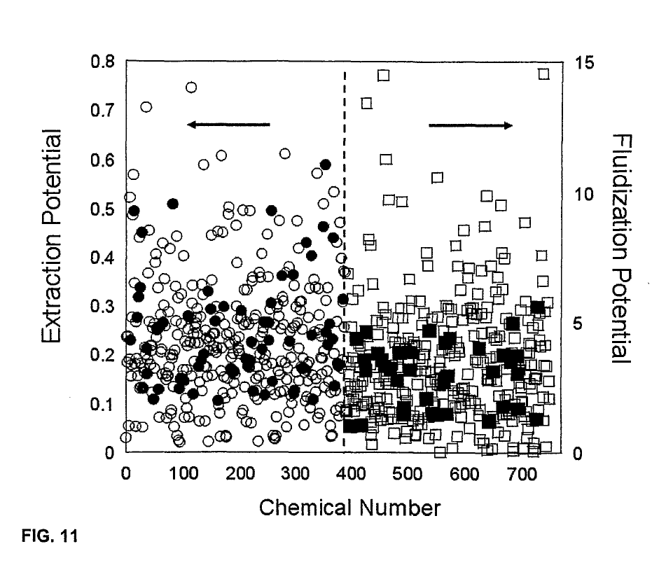

FIG. 10 is a flow diagram showing a sequence of steps that may be applied to screen the performance of molecules as penetration enhancers according to a preferred embodiment of the invention. The sequence begins at step 102 where a starting set of molecules is provided. The starting set of molecules may be any suitable set of molecules, provided that the set contains at least one molecule. In a preferred embodiment of the invention one or more of the molecules in the starting set are structurally related to known CPEs. Structurally related analogs of known CPEs may be constructed using appropriate software. For example, software such as the Cerius² Analog Builder (Accelrys, San Diego, Calif. www.accelrys.com) automatically construct large sets of analog molecules by systematically substituting user-specified groups for up to three hydrogen atoms on a parent structure. The choice of substituents to add to the molecule may, for example, be guided by previous work on CPEs such as the QSAR relartionships summarized by Kanikkannan et al. Kanikkannan et al. (2000). In two other preferred embodiments of the invention the molecules in the starting set are structurally unrelated to known CPEs and are organic molecules.

At step 104 descriptors for the current set of molecules are obtained. The descriptors may be obtained, for example, from the scientific literature or retrieved from electronic databases. In a preferred embodiment of the invention, the descriptors are calculated based on the molecular structures of the molecules in the set. Several commercial software packages provide capabilities for calculation of descriptors including, for example, Cerius² (Accelrys, San Diego, Calif. www.accelr s.com) and Molecular Modeling Pro™ (MMP) by ChemSW® (Fairfield, Calif. www.chemsw.com). Preferably, where relevant, descriptor values are calculated using molecular structures that have been minimized, for example, using forcefields such as those available in the aforementioned software packages, Particularly preferred descriptors for calculation include solubility parameters, cohesive energy densities and water-octanol partition coefficients (logP). In a preferred embodiment of the invention it is preferred that the values of logP are computed following the methods described by (i) Viswanadhan et al., (ii) Hansch et al., (iii) Bodor et al. and (iv) Moriguchi et al. and the average of the three closest values used in subsequent analysis. Viswanadhan et al. (1989); Hansch et al. (1979); Bodor et al. (1997); Moriguchi et al. (1992); Moriguchi et al. (1994). In another preferred embodiment hydrogen bonding (δₕ), polarity (δₚ) and dispersion (δ_{d}) are calculated using the 3-D solubility parameters following the methods of (i) Hansen (proprietary algorithm of ChemSW® accessible through Molecular Modeling Pro™), (ii) van Krevelen and Hoftyzer and (iii) Hoy, and an average of two closest values was used in subsequent analysis. van Krevelen (1990); Hoy (1970).

At steps 106, 108 and 110 the descriptor information obtained in step 104 is used to compute (i) the ability of the molecules in the set to enhance transport assuming the molecule behaves as a fluidizer, (ii) the ability of the molecule to enhance transport assuming the molecule behaves as an extractor and (iii) the irritation potential of the molecule. In a preferred embodiment of the invention the ability of the molecule to enhance transport assuming a fluidizer is computed according to a quantity, which may be termed fluidization potential (FP), defined through

FP=log P.

In another preferred embodiment of the invention the ability of the molecule to enhance transport assuming an extractor is computed according to a quantity, which may be termed extraction potential (EP), defined through

$$EP = \frac{{δ}_{h}}{\sqrt{{δ}_{p}^{2} + {δ}_{h}^{2} + {δ}_{d}^{2}}} .$$

In yet another preferred embodiment of the invention the irritation potential (IP_{Desciptor}) is computed using,

$${IP}_{Desciptor} = \frac{{δ}_{h}}{{δ}_{p}} .$$

At step 112 the performance of the current set of molecules as chemical penetration enhancers is analyzed to identify some leading candidates. For example, the performance of the molecules can be compared against known CPEs. FIG. 11 is a plot of extraction potential and fluidization potential for a series of molecules which includes both known CPEs and molecules not previously known to be CPEs and shows one approach to such analysis.

At step 114 a decision is made as to whether the performance of the leading candidates justifies in vitro testing of one or more of the lead candidates. Many factors may be considered in taking this decision including comparisons of the performance of the lead candidates versus known CPEs, the difficulty of obtaining samples of the lead candidate molecules (e.g. by acquiring from chemical suppliers or by chemical synthesis), toxicity information on the lead candidates and so forth. If a decision is made to proceed with in vitro testing, the work flow may proceed to step 116. In a preferred embodiment of the invention the ability of the lead candidates to enhance the transport of molecules is validated using devices such as Franz diffusion cells in combination with appropriate actives and irritation potential examined using in vitro skin cultures such as EpiDerm™. In another preferred embodiment the performance of the CPEs is evaluated using IR spectroscopy by examining the changes in the area of under the symmetric methylene peak and the changes in the Amide I band of a sample of stratum corneum before and after treatment with a formulations containing lead candidates. Conversely, if it is decided that the performance of the leading candidates does not justify in vitro testing the workflow proceeds to step 118, and a new set of molecules provided. In a preferred embodiment of the invention the some, or all, of the lead candidates determined at step 112 are modified by making substitutions believed likely to improve the performance of the molecules as CPEs. In another preferred embodiment of the invention, a new set of molecules that bears no special relationship to the lead candidates determined at step 112 is provided at step 118.

The present invention has numerous embodiments in addition to those following the work flow presented in FIG. 10. For example, step 18 may be omitted and the work flow terminated if no lead candidates justify in vitro testing. Similarly, one, or even two, of the steps 106, 108 and 110 may be omitted from the workflow if desired. Alternatively, both step 114 and 118 may be omitted and the process may instead proceed directly to in vitro testing of lead CPE candidates at step 116. Step 116 may also be omitted and the testing of lead CPE candidates may proceed directly to animal or human testing, if desired.

It will also be appreciated that the present invention may be applied to problems outside the field of discovery of new CPEs. For example, the ability to determine the irritation potential of compounds using descriptors may be beneficially applied in the design and optimization of drugs and other actives. Similarly, this ability may be used in selection and design of excipients for formulations intended for application to skin and mucosa.

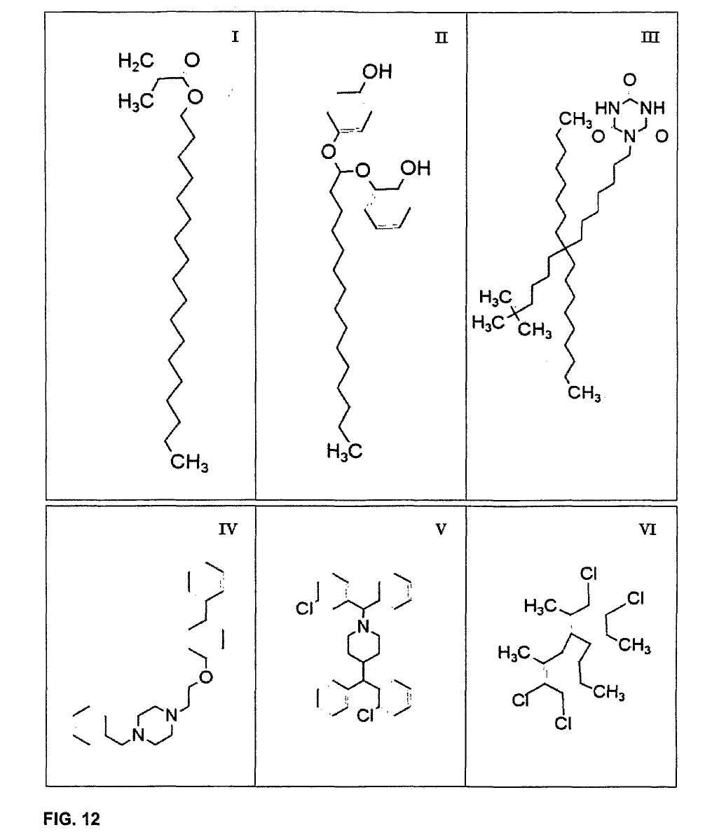

A practical application of this in silico approach to discovering CPEs in provided in Example 4 herein. The structures of molecules that have been discovered to have interesting CPE properties using the in silico approach are depicted in FIG. 12. These molecules of the present invention may be utilized in any situation where enhancement of transport of an active into the skin of an animal or human is desired. In preferred embodiments of the invention the molecules are used to enhance transport of actives for transderrnal delivery and topical delivery. Structural analogs of these molecules which substantially preserve the ER/IP values of the molecules depicted in FIG. 12 may also be utilized for such purposes in the context of the present invention.

### EXAMPLE 1

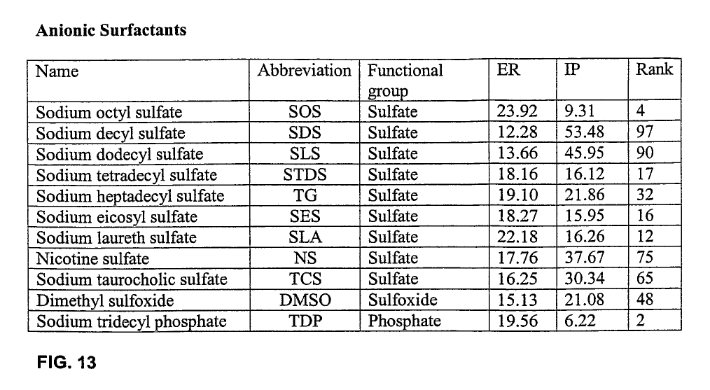

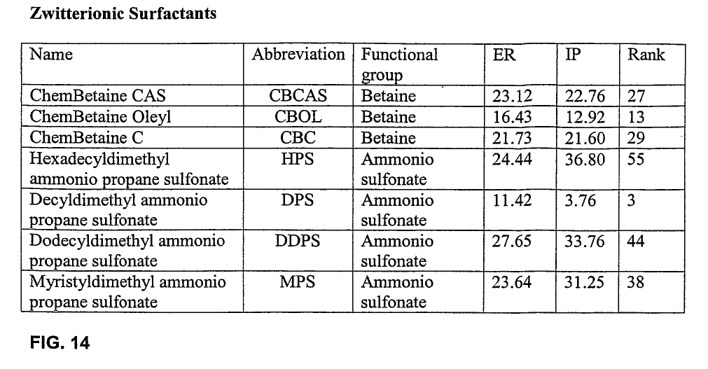

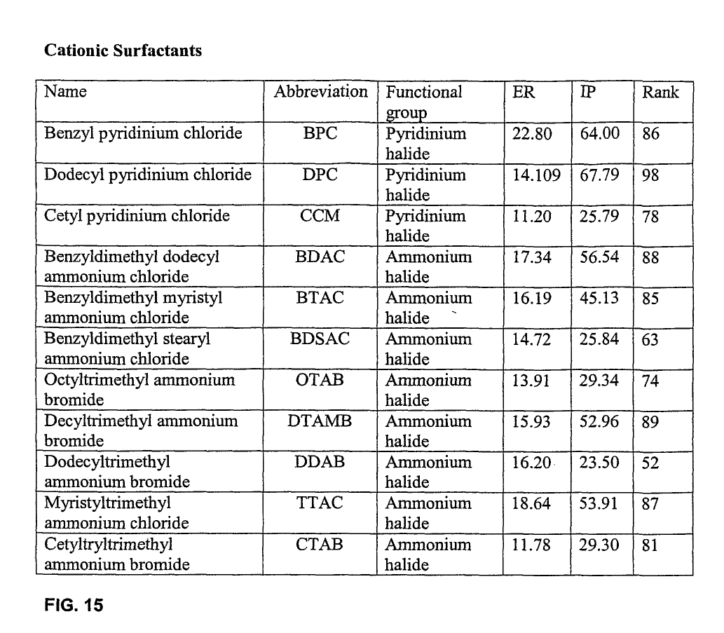

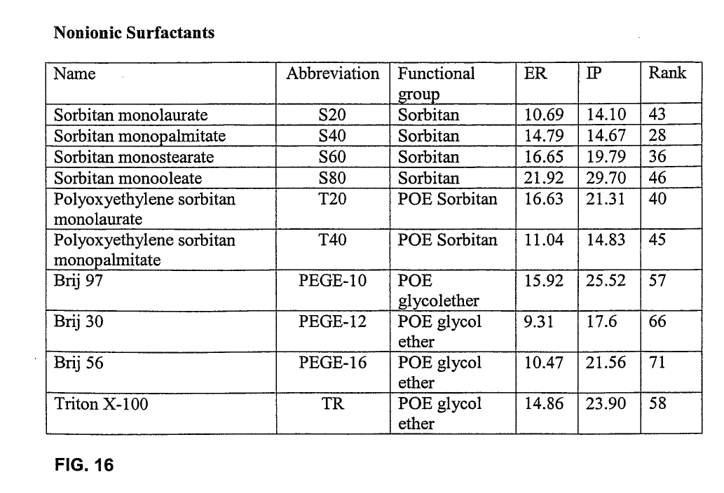

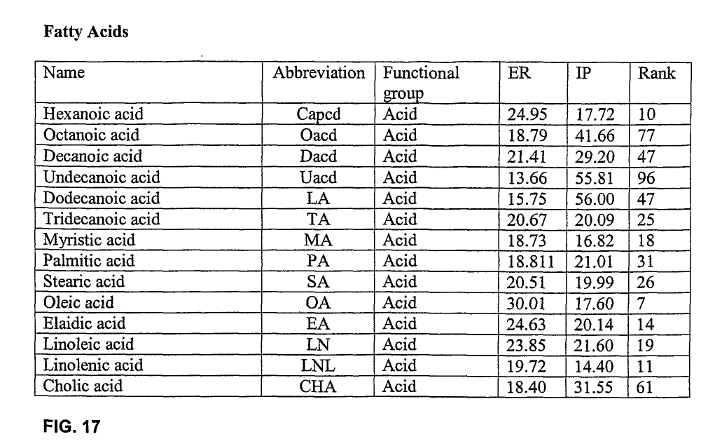

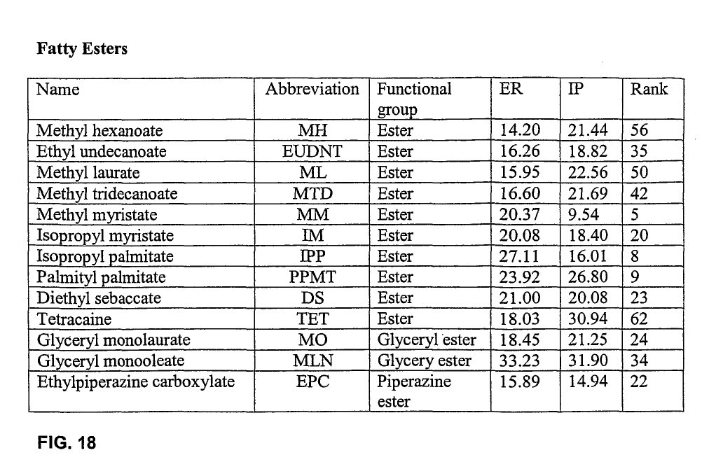

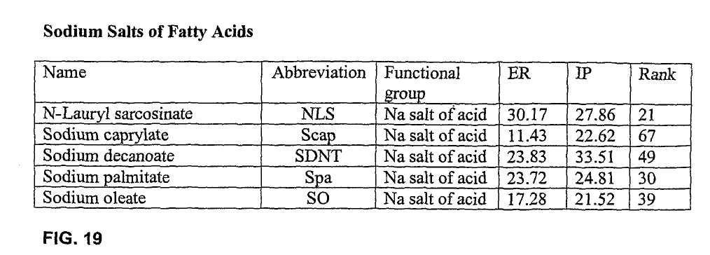

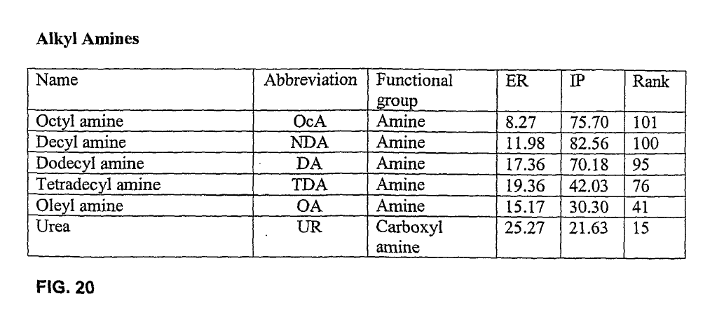

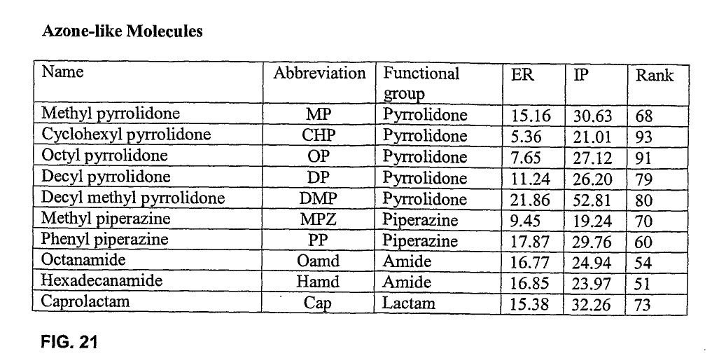

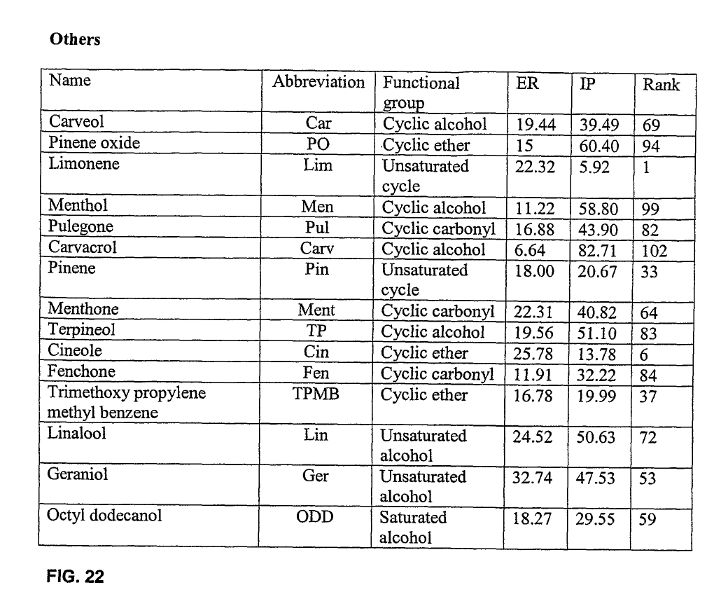

102 CPEs were chosen from following ten categories: (i) anionic surfactants (AI); (ii) cationic surfactants (CI); (iii) zwitterionic surfactants (ZI); (iv) non-ionic surfactants (NI); (v) fatty acids (FA); (vi) fatty esters (FE); (vii) alkyl amines (FM); (viii) azone-like compounds (AZ); (ix) sodium salts of fatty acids (SS); and (x) others (OT). The chemical names and abbreviated names of the 102 CPEs, are provided in FIG. 13 to FIG. 22. These chemicals provide a relatively diverse collection of molecules from the known space of CPEs, and include several well-known CPEs from the transdermal drug delivery literature. Formulations containing 1.5% (wt/vol) of each CPE were prepared in a 1:1 EtOH:PBS (ethanol:phosphate buffered saline) solvent.

Irritation potential of formulations was estimated using EpiDerm™ (MatTek Corporation, Ashland, Mass. www.mattek.com), a cell culture of normal human derived epidermal keratinocytes. EpiDerm™ cultures were stored and handled according to the standard protocol MTT-ET-50 supplied by MatTek Corporation. To study the effect of the test formulations on cell viability, cell cultures were exposed to 10 μl of the test formulation consisting of 1.5% (wt/vol) of each CPE in a 1:1 EtOH:PBS solvent for 4 hours. Each test formulation was analyzed in duplicate. The cell cultures were then handled as per the MTT-ET-50 protocol. The optical absorbance data from the extracted samples was then used to calculate the percentage cell viability as recommended in the protocol. Based on the cell viability, the irritation potential, IP, was defined as follows:

$$IP = 100 ( 1 - \frac{\% cell viability with the formulation}{maximum \% cell viability} ) .$$

1% (wt/vol) Triton X-100 dissolved in water was used as the positive control and 1:1 PBS:EtOH was used as negative control. The irritation potentials measured according to this protocol of the 102 CPEs studied in this example are reported in the columns headed “IP” in the tables in FIG. 13 to FIG. 22.

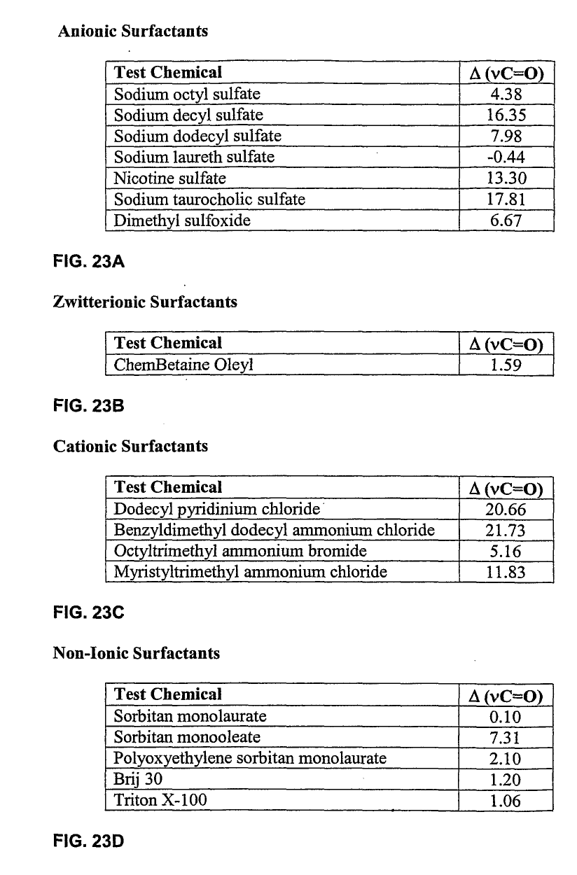

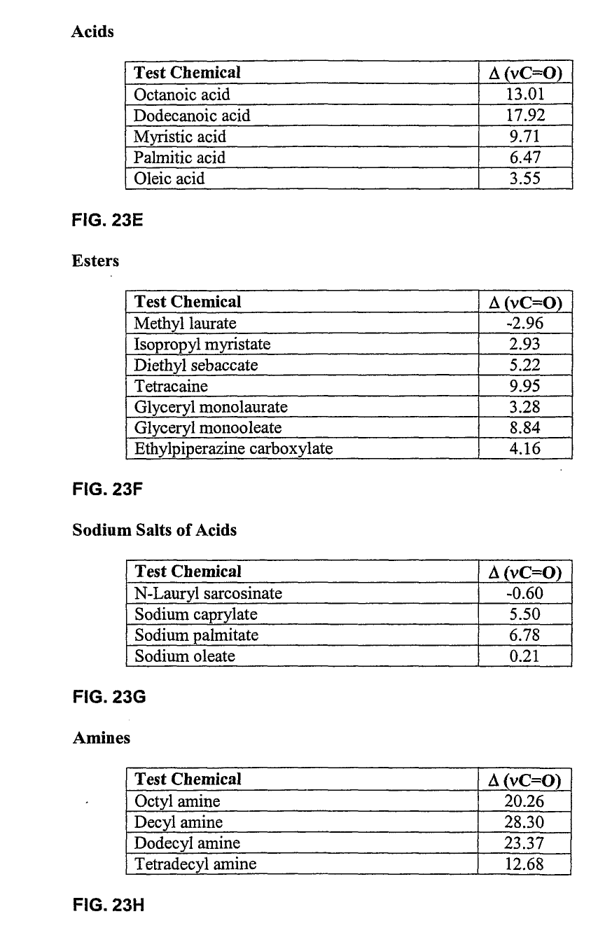

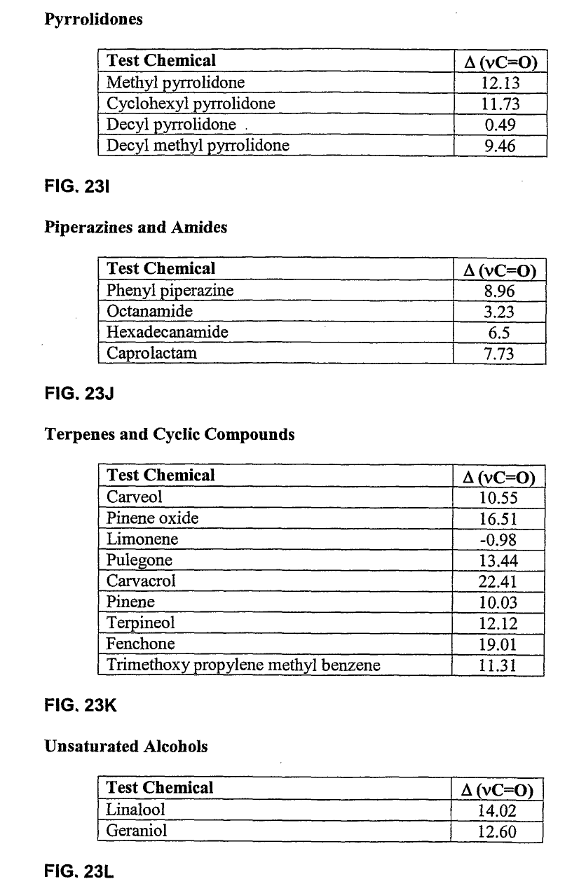

The interactions of 56 of the CPEs, selected from the original group of 102 CPEs, with the stratum corneum were also studied using FTIR. The CPEs selected for further study are shown in FIG. 23. Each CPE formulation was studied in triplicate to assess its effect on the stratum corneum. Each piece of stratum corneum prior to CPE application was used as its own control and accordingly interferograms were recorded on the stratum corneum samples before and after treatment.

Samples of stratum corneum were prepared as follows. Porcine skin that had been previously harvested from Yorkshire pigs and stored at −70° C. was defrosted at room temperature. Porcine epidermis was isolated from the dermis in full thickness skin using heat stripping. Kligman et al. (1963); Simon et al. (2000). Full thickness skin was dipped in water at ˜62° C. for 90 seconds and blotted dry. The epidermis was carefully peeled from the dermis using forceps taking care that no damage was done to the intact epidermal membrane. The isolated epidermis was then floated over 0.25% (wt/vol) trypsin solution (Sigma Aldrich, St. Louis, Mo. www.sipmaaldrich.com) overnight at room temperature to digest the epidermal matrix of keratinocytes. The residual stratum corneum film was then washed with PBS and dried at room temperature for 48 hrs. At this point the stratum corneum was essentially free of hair and other epidermal debris. The stratum corneum was then cut into square pieces with area of approximately 1.5 cm×1.5 cm. The IR spectrum of each sample was recorded.

Each stratum corneum sample was then incubated with the CPE formulation for 24 hours at room temperature by immersing the samples in 500-1,000 μl of the formulation contained in screw-top vials. Tops were placed on the vials during the incubation period to minimize evaporation. The CPE formulations contained 1.5% wt/vol CPE were prepared using deuterated solvents (1:1 EtOD:D₂O). At the end of incubation period the stratum corneum samples were removed from the CPE formulation and were rinsed thoroughly with 1:1 EtOD:D₂O to remove any excess chemical penetration enhancer residing on the SC surface. The SC pieces were then dried at room temperature for 48 hrs at the end of which interferograms were recorded again.

Spectra were recorded using a Nicolet Magna 750 spectrometer (Thermo Electron Corporation, Waltham, Mass. www.thermo.com) at a resolution of 2 cm⁻¹ and were averaged over 100 scans to reduce noise in the spectrum. Spectra were accumulated in absorbance mode. The stratum corneum samples were held in a Demountable Cell Kit by Spectra-Tech: part # FT04-036. The spectra were smoothed and base line corrected and saved in the CSV (comma separated value) format for further analysis.

The IR spectra information stored in the CSV format was exported to the Origin software (OriginLab Corporation, Northampton, Mass., www.originlab.com). The Amide I bands of the spectra, which are sensitive to protein conformation and that generally fall in the 1700-1600 cm⁻¹ region of the IR absorption spectrum, were characterized. The spectra Amide I bands of the spectra were decomposed by fitting of Gaussians by standard statistical methods of peak fitting in Origin. Center, height, bandwidth, offset and area values were recorded for each of the deconvoluted peaks. Byler et al. (1986); Krimm et al. (1986). The deconvolution procedure was applied to the spectrum of each SC sample obtained before and after treatment with CPE.

The irritation response of CPEs measured with EpiDerm™ was discovered to correlate with changes in the Amide I band (1700-1600 cm⁻¹) in the IR absorption spectrum of the stratum corneum. Examples of deconvoluted peaks of the Amide I band of a stratum corneum IR spectrum before and after the treatment with a formulation containing 1.5% wt/vol lauric acid in 1:1 EtOD:D₂O are shown in FIG. 4 and FIG. 5, respectively. In the IR spectrum of untreated SC the absorbance in the 1660-1650 cm⁻¹ region is at maximum indicating an abundance of α-helical conformations in stratum corneum proteins as may be seen in FIG. 4. Treatment with lauric acid decreases the relative contribution of the α-helical structures to the Amide I band compared to the untreated region of the same sample. Contributions from other secondary structures, β sheets, random coils and antiparallel β sheets and turns, in contrast, increase compared to the corresponding regions of the untreated sample.

The change in the integrated absorbance of the deconvoluted spectrum in the region of 1660-1650 cm⁻¹, (Δ(νC═O)), was calculated as described above and was discovered to correlate well against the IP values assessed using EpiDerm™. Values of Δ(νC═O) are reported in FIG. 23. FIG. 6, is a plot of Δ(νC═O) against irritation potential measured with EpiDerm™. It may be seen that these parameters are strongly correlated (r²=0.7).

Without being bound by theory, the effects of the formulations on the Amide I peak in the FTIR spectra may be interpreted as arising from denaturing of the stratum corneum proteins. Formulation constituents that gain access to the interior of the corneocytes may prompt unfolding of the stratum corneum proteins thereby changing their conformation to other less rigid secondary structures. Such conformational changes are characteristic of protein unfolding or denaturing. Byler et al. (1986). Decreases in the C═O peak intensity (1650 cm⁻¹) showed a tendency to be, proportionally, accompanied by an increase in the intensity of N-D bending vibrations peak (1440-1450 cm⁻¹) in the Amide II band of the FTIR spectrum. This peak, arising from hydrogen-deuterium (H-D) exchange between proteins and formulations, provides evidence that irritating chemicals indeed breach the comified envelope and expose the amide bonds in the SC proteins to EtOD:D₂O. Extensive H-D exchange between proteins and solvents has been typically associated with unfolding. Kluge et al. 1998.

### EXAMPLE 2

The potencies of the 102 CPEs introduced in Example 1 were quantified by skin conductivity enhancement ratios (ER) using the methods described in International Application Number PCT/US01/26473 published under International Publication Number WO 02/16941 A2 and International Application Number PCT/US2004/023634 published under International Publication Number WO 2005/009510 A1. Skin conductivity enhancement ratios provide a convenient assay for assessing the effects of a formulation on the barrier properties of skin.

Enhancement ratios were measured using an apparatus (the INSIGHT apparatus) which consisted of a polycarbonate plate that served as the receptor plate and a Teflon® plate that served as the donor plate. Each plate was 12.7 mm thick. The donor contained a square matrix of 100 wells (each 3 mm in diameter) that served as individual donor compartments. The center-to-center distance between the donor compartments was 6 mm. A matching matrix of 100 wells in the Teflon® plate served as individual donors. The receptor wells were filled with PBS to keep the skin hydrated over the entire duration of the experiment (24 hrs). Skin that had been previously harvested from Yorkshire pigs according to the methods described by Mitragotri et al. and stored at −70° C. was thawed at room temperature prior to each experiment. Mitragotri et al. (2000). The skin was then placed between the two plates with the stratum corneum facing the donor plate. Donor and receptor plates were clamped together using 4 bolts and wing nuts and the level of fluid in each well was followed to ensure there was no leakage of formulation between adjacent wells. The skin was incubated with 85 μL of each test formulation in the donor wells for a period of 24 hrs with each formulation being repeated in at least four wells.

Skin impedance in each well was recorded using two electrodes. One electrode was inserted into the dermis and served as a common electrode while the second electrode was placed sequentially by hand into each donor compartment. An AC signal, 100 mV RMS at 100 Hz, was applied across the skin with a waveform generator (Agilent 33120A, Palo Alto, Calif. www.agilent.com). Conductivity measurements were performed using a multimeter (Fluke 189, Everett, Wash. www.fluke.com) with a resolution of 0.01 μA Current measurements were performed at two time points, time t=0 (I₀) and time t=24 hrs (I₂₄). The AC signal was only applied while conductivity measurements were being made. The conductivity enhancement ratio (ER) for each formulation was then calculated by taking the ratio of skin conductivities at 24 and 0 hours according to

$$ER = \frac{{I}_{24}}{{I}_{0}}$$

Conductivity enhancement ratios of the 102 CPEs of the present example are provided in the columns titled “ER” in the tables shown in FIG. 13 to FIG. 22.

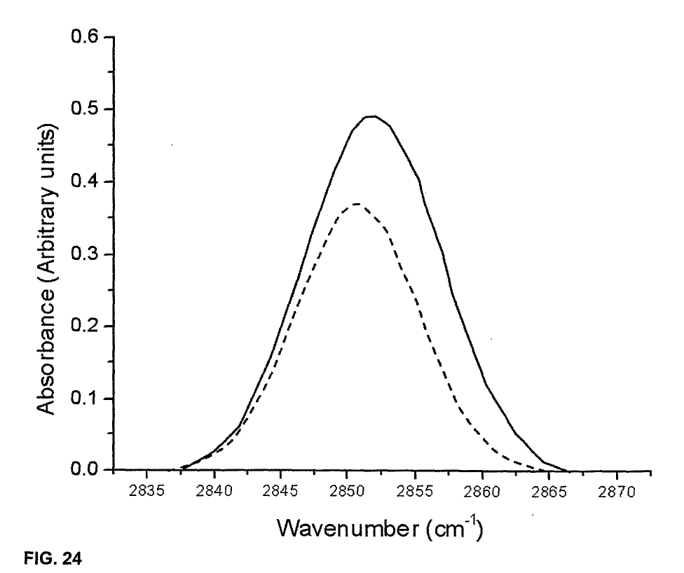

ER values were discovered to correlate with the changes in the integrated absorbance of the symmetric methylene stretching modes, ν_{sym}CH₂, of FTIR spectra of the stratum corneum before and after treatment with CPE measured according to the FTIR protocol set out in Example 1. FIG. 24 shows sample IR spectra of the symmetric methylene stretching mode (ν_{sym}CH₂) at 2850 cm⁻¹. The dashed curve and solid curve show the absorbance of the ν_{sym}CH₂ mode of a stratum corneum sample before and after treatment, respectively, with formulation containing 1.5% wt/vol Lauric acid in 1:1 D₂O:EtOD. FIG. 7 is a plot of conductivity enhancement ratio against change in integrated absorbance of methylene stretching; Δ(ν_{sym}CH₂) produced by formulations containing the 56 CPEs listed in FIG. 23. Δ(ν_{sym}CH₂) was obtained by analyzing the spectra whose collection was described in Example 1 using

Δ(ν_{sym}CH₂)=area before treatment−area after treatment.

Error bars in FIG. 7 correspond to N=3. It can be seen in FIG. 7 that CPEs may be divided into two categories, depending on whether Δ(ν_{sym}CH₂) is positive or negative. CPEs in each category show a separate linear correlation with ER (r²=0.67 for Δ(ν_{sym}CH₂) negative; r²=0.53 for Δ(ν_{sym}CH₂) positive).

### EXAMPLE 3

Molecular descriptors were calculated for the 56 CPEs listed in FIG. 23 using Molecular Modeling Pro™ (MMP) by ChemSW® (Fairfield, Calif. www.chemsw.com). Molecular structures were drawn using the interface provided by MMP. Structures were minimized to represent the 3-D structure in the lowest energy configuration using the default minimization procedure in MMP.

Thermodynamic properties that were calculated included:
(i) Log octanol-water partition coefficient (LogP);
(ii) Hydration number after McGowan; McGowan (1990);
(iii) Hydrophilic lipophilic balance (HLB);
(iv) Solubility parameters, hydrogen bonding (δₕ (J/cc)^{1/2}), polarity (δₚ (J/cc)^{1/2}) and dispersion (δ_{d} (J/CC)^{1/2});
(v) Energy of cohesion, calculated using the method outlined by Fedors (EC, J/mol); Fedors (1974)
(vi) Surface tension (ST, dynes/cm);
(vii) Water solubility (mol/L) after Klopman; Klopman et al. (1992);
(vii) Hydrogen bond acceptance (HBA) and hydrogen bond donation (HBD);
(viii) Mean water of hydration (MWH);
(ix) Hydrophilic surface area (HAS, cm²/mol);
(x) Percentage hydrophilic surface (HS); and
(xi) Polar surface area (PSA, Å²).

Physical properties that were calculated included:
(i) Atom based Molar Refraction (MR);
(ii) Molar volume (MV, cc/mol);
(iii) Molecular volume (MV, Å³);
(iv) Molecular weight (MW, Da);
(v) Molecular surface area (SA, Å²);
(vi) Molecular length (ML, Å);
(vii) Molecular width (MWD, Å);
(viii) Density (g/cc);
(ix) Dipole moment (Debye); and
(x) Molecular charge.

Octanol water partition coefficients was calculated using 4 independent methods: (i) atom based logp (ALogP) after Viswanadhan et al., (ii) fragment addition logp (FLogP) after Hansch and Leo, (iii) QLogP after Bodor and Buchwald and (iv) Moriguchi LogP. Viswanadhan et al. (1989); Hansch et al. (1979); Bodor et al. (1997); Moriguchi et al. (1992); Moriguchi et al. (1994). The average of the three closest values was used for analysis. Hydrogen bonding, polarity and dispersion were calculated using the 3-D solubility parameters following the methods of (i) Hansen (proprietary algorithm of ChemSW®), (ii) van Krevelen and Hoftyzer and (iii) Hoy. van Krevelen (1990); Hoy (1970). As with calculated logP values, instead of using any one independent method for calculation of solubility parameters an average of the two closest values was used. A total of 35 different parameters were calculated for each CPE.

A correlation matrix was run on all variables to eliminate redundant variables in an attempt to discover descriptors that correlated with changes in the FTIR spectra discussed in Example 1 and Example 2. Fluidization potential of CPEs (as quantified by an increase in the integrated absorbance of ν_{sym}CH₂ peak) was discovered to correlate with CPE hydrophobicity quantified in terms of LogP, the octanol-water partition coefficient as shown by the open circles in FIG. 8, (r²=0.86). On the other hand, the extraction potential of CPEs (as quantified by a decrease in ν_{sym}CH₂ peak area) was discovered to correlate with the ratio of hydrogen bonding (δₕ) to square root of cohesive energy density (E_{C}=δₚ²+δₕ²+δ²_{d}) (FIG. 8, closed circles, r²=0.54). The irritation potential of CPEs (as quantified by Δ(νC═O)) was discovered to correlate with the ratio of hydrogen bonding (δₕ) to polar forces (δₚ) for both extractors and fluidizers as shown in FIG. 9 (r²=0.78).

Having discovered molecular descriptors for ER and IP a descriptor for the overall quality of a CPE (ER/IP) may be defined as follows:

$$\begin{matrix}\frac{ER}{IP} {\vert}_{extractors} ∝ \frac{{δ}_{p}}{\sqrt{{δ}_{p}^{2} + {δ}_{h}^{2} + {δ}_{d}^{2}}} , and & EQ . 1 \\ \frac{ER}{IP} {\vert}_{fluidizers} ∝ Log P ( \frac{{δ}_{p}}{{δ}_{h}} ) . & EQ . 2\end{matrix}$$

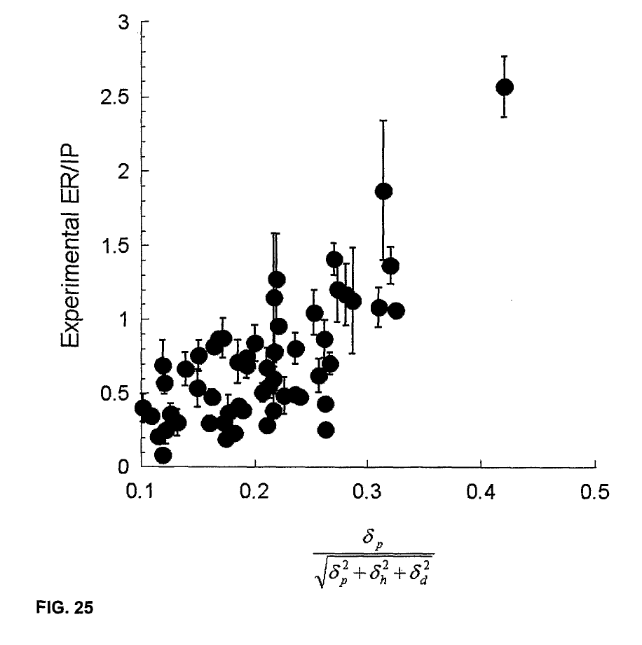

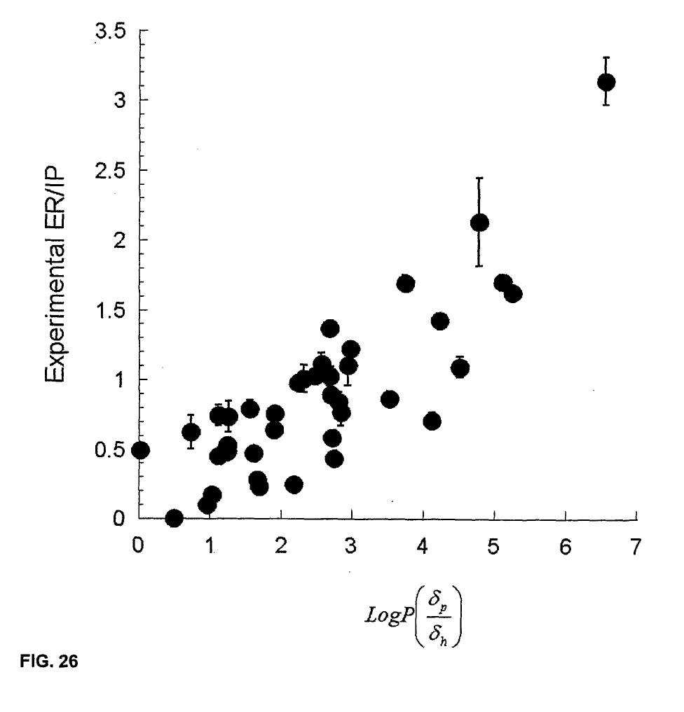

FIG. 25 and FIG. 26 are graphs showing the correlation between measured values of ER/IP (as determined in Example 1 and Example 2) against the molecular descriptors for ER/IP proposed in EQ. 1 and EQ. 2 for all fluidizers (r²=0.84) and extractors (r²=0.73) for the 56 CPEs considered here.

### EXAMPLE 4

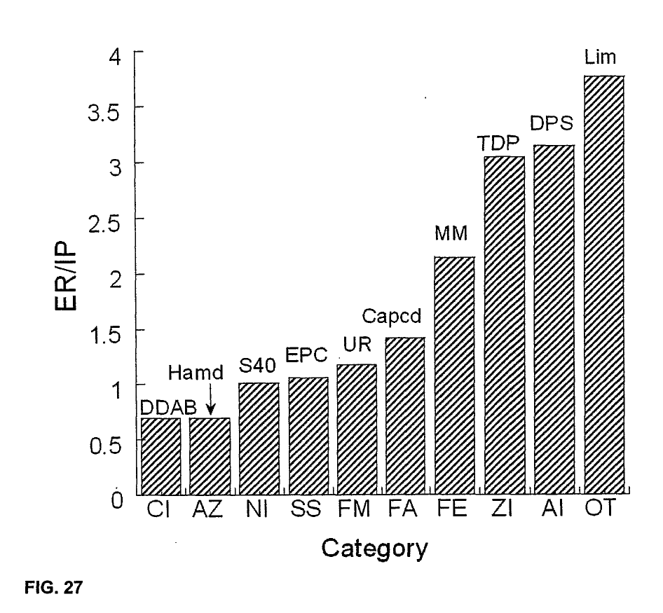

ER/IP values for the 102 CPEs introduced in Example 1 were computed using IP and ER values determined in Example 1 and Example 2, respectively. The ranking of each CPE, as quantified by its ER/IP, value is reported in FIG. 13 to FIG. 22. FIG. 27 is a bar graph showing the highest ER/IP value for CPEs in each of the chemical classes of CPEs shown in FIG. 13 to FIG. 22 (the names of the CPEs shown in FIG. 27 follow the abbreviation scheme introduced in FIG. 13 to FIG. 22). The 10 best-of-class CPEs identified in FIG. 27 were selected for in silico mutation. These initial starting CPEs may be termed wild-type CPEs. Limonene, the molecule with the highest calculated ER/IP value from the entire set of 102 CPEs was computed to have an ER/IP value of about 4.

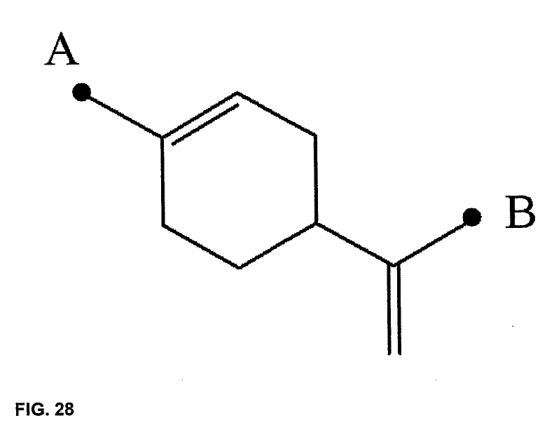

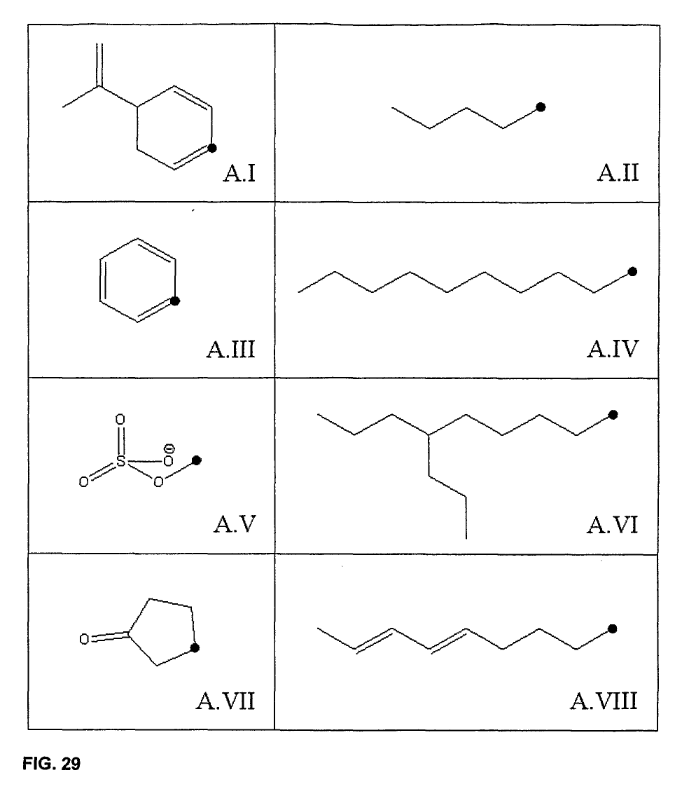

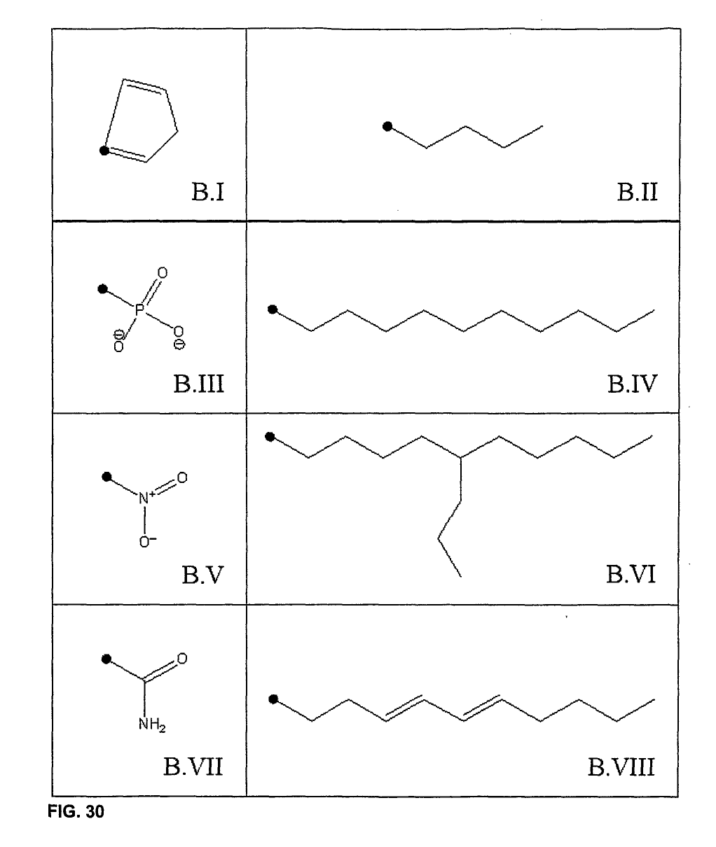

Mutations were performed on the wild-type CPEs by substituting one of its chemical functional groups to generate a library of putative CPEs. Putative CPEs, created by substituting a functional group on a wild type CPE or another putative CPE, may be termed mutant CPEs, or simply, mutants. For example, FIG. 28 shows the structure of limonene. Mutant molecules were derived by defining two substitution points, labeled A and B in FIG. 27, on the molecule. The fragments shown in FIG. 29 and FIG. 30 were then substituted sequentially in silico at the points A and B, respectively, on the limonene molecule. By this procedure, the 16 fragments shown in FIG. 29 and FIG. 30 led to 16 putative CPEs based on mutations of the limonene structure. The choice of functional groups to attach to molecules was guided in part by intuition (e.g. good fluidizers tend to have fatty acid tails, where as good extractors often have large polar head groups).

The mutant CPEs, along with their parent wild-type CPEs, were screened using EQ. (1) and EQ. (2) introduced in Example 3 to identify safe and potent extractors as well as fluidizers. The molecules in the library were allowed to evolve in an iterative fashion by selecting candidates showing best ER/IP values from each generation and making further substitutions in an attempt improve performance. Approximately 325 different mutant CPEs were studied. Descriptors were calculated using Molecular Modeling Pro™, as described above.

The left hand panel of FIG. 11 is a graph showing as the y axis the relative performance (as measured by ratio of enhancement ratio to irritation potential) for the wild-type (closed circle) and mutant CPEs (open circle), assuming the molecules perform as extractors, modeled using EQ. 1 above. The left hand panel of FIG. 11 is a graph showing the relative performance for the wild-type (closed square) and mutant CPEs (open square) assuming the molecules perform as fluidizers modeled as log P. The library of mutant CPEs contained molecules whose fluidization and extraction potential, as assessed by the correlations introduced in Example 3, were improved over that achieved by the starting wild-type CPEs.

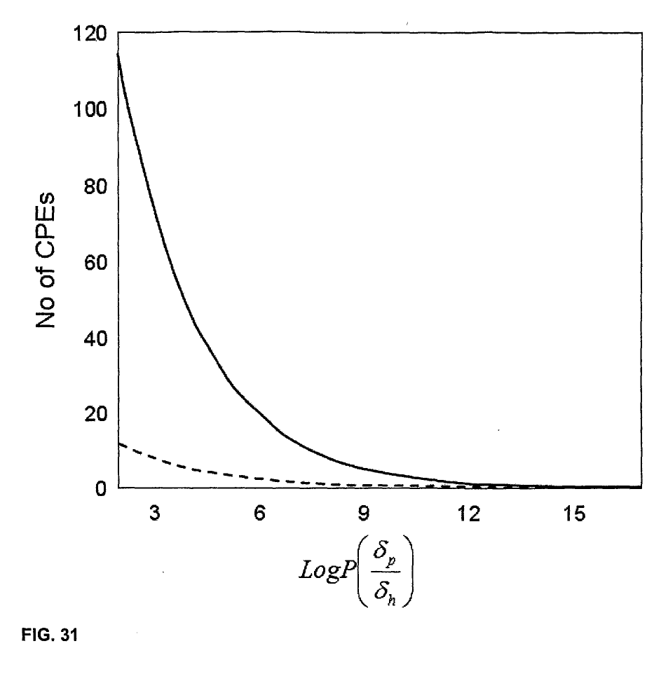

FIG. 31 is a graph showing the y axis the number of starting wild-type (dotted line) and resulting mutant (solid line) CPEs in the study that exceed a threshold FP/IP_{Descriptor} against FP/IP_{Descriptor} or on the x axis. The FP/IP_{Descriptor} values of the population of mutant CPEs compared favorably to those of the original pool of 102 enhancers. In the original pool of 102 enhancers, about 9 fluidizers exhibited FP/IP_{Descriptor} better than 3.8. An FP/IP_{Descriptor} value of 3.8 corresponds to that of oleic acid (OA) a commonly used fluidizer CPE in transdermal drug delivery literature. The proportion of mutant fluidizers with ER/IP values greater than this threshold increased by a factor of 12 and 110 mutant fluidizers, out of the pool of approximately 325 mutant fluidizers, showed FP/IP_{Descriptor}>3.8. Chemical structures of some of the lead mutants are shown in FIG. 12.

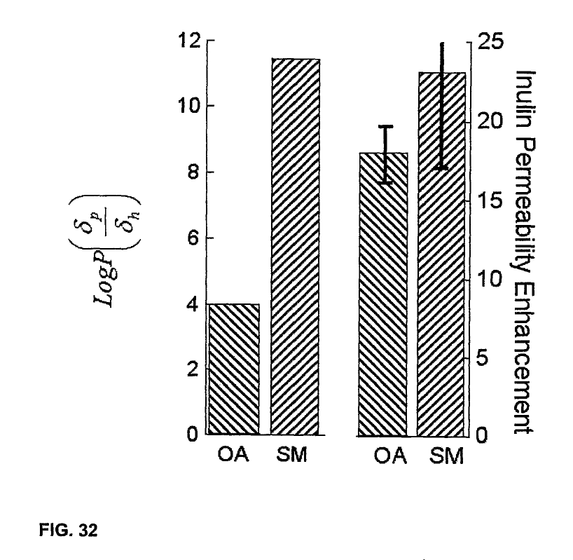

One of the lead mutants, stearyl methacrylate (SM), was found to be commercially available. Its ER/IP value was determined experimentally using EpiDerm™ and the INSIGHT apparatus according to the procedures set out in Example I and Example 2. The FP/IP_{Descriptor} value of SM was measured to be about 3 times higher than oleic acid as may be seen in the left-hand panel of the bar chart of FIG. 32.

The ability of SM also to enhance the delivery of a model macromolecule, inulin across the skin in vitro was measured using Franz diffusion cells. Concentration changes of the inulin due to transport in the Franz diffusion cell was measured using ³H-labeled inulin acquired from American Radiolabeled Chemicals of St. Louis, Mo. (www.arc-inc.com). Formulations containing 1.5% wt/vol of the CPEs oleic acid and stearyl metbacrylate, respectively, in a vehicle of 1:1 PBS:EtOH were prepared. Transport of inulin in these CPE-containing formulations was compared to that achieved with 1:1 PBS:EtOH. Inulin was added to the formulations at a concentration of 10 μCi/ml. The resulting formulations were placed in the donor well of Franz cells and the contents of the receiver wells were sampled periodically for a period of 96 hours to monitor transport. FDCs utilized in the experiments had a diameter of 16 mm and receiver volume of 12 ml. Small stir bars and Ag/AgCl disk electrodes (model number E242 acquired from In Vivo Metric, Healdsburg, Calif. (www.invivometric.com) were added to the receiver chamber, the disk electrode allowing skin conductivity to be measured as the experiment proceeded. The FDC receiver chambers were filled with PBS and adequate measures were taken to prevent inclusion of air in the receiver chamber. Thawed pig skin, harvested from Yorkshire pigs and stored at −70° C. immediately after procurement until the time of experiments using the methods described by Mitragotri et al. was mounted on the diffusion cell using a clamp with the stratum corneum side facing the donor well. Mitragotri et al. (2000). The concentration of the radiolabeled test molecule was measured using a Packard Tri-Carb 2100 TR scintillation counter. FDC measurements were repeated several times for each test molecule to ensure statistically meaningful results. In order to confirm that detected radioactivity was a result of transport of the test molecules and not from tritiated water that may have resulted from tritium exchange, receiver samples were desiccated and analyzed for radioactivity. No substantial differences in radioactivity were observed between native and desiccated receiver samples.

Inulin permeability enhancement for formulations containing stearyl methacrylate and oleic acid compared to PBS:EtOH are reported in bar chart in the right-hand panel of FIG. 32.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. Also as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

### CITATIONS

Asbill, C. S., El-Kattan, A. F. and Michniak, B., Enhancement of transdermal drug delivery: chemical and physical approaches. Crit. Rev. Ther. Drug Carrier Syst. 2000 17:621-658.
Bauerova, K., Matusova, D. and Kassai, Z., Chemical enhancers for transdermal drug transport. Eur. J. Drug Metab. Pharmacokinet. 2001 26:85-94.
Bellamy, L. J., The infra-red spectra of complex molecules, 2nd ed.; Methuen: London, 1958 p 380.
Berger, R. S, and Bowman, J. P., A reappraisal of the 21-day cumulative irritation test in man. J. Toxicol.—Cut. & Ocular Toxicol. 1982 1:109-115.
Berti, J. and Lipsky J. J., Transcutaneous drug delivery: A practical review. Mayo Clinical Proceedings, 1995. 70:581-586.
Bodor, N. and Buchwald, P., A Molecular size based approach to estimate partition properties for organic solutes. J. Phys. Chem. B 1997 101:3404-3412.
Bommannan, D., Potts, R. O. and Guy, R. H., Examination of the effect of ethanol on human stratum corneum in vivo using infrared spectroscopy. Journal of Controlled Release 1991 16:299-304.
Bos, J. D., Meinardi, M. M. H. M., The 500 Dalton rule for the skin penetration of chemical compounds and drugs. Exp. Dermatol. 2000. 9:165-169.
Bouwstra, J. A., The skin, a well organized membrane. Colloids and Surfaces. A, 1997. 123-124:403-413.
Byler, D. M. and Susi, H., Examination of the secondary structure of proteins by deconvolved FTIR spectra. Biopolymers. 1986 25:469-487.
Draize, J., Woodward, G. and Calvery, H. J. Methods for the study of irritation and toxicity of substances applied topically to the skin and mucous membranes. Pharmacol. Expt. Thera. 1944 82:377-390.
Eun, H. and Suh, D. J., Comprehensive outlook of in vitro tests for assessing skin irritancy as alternatives to Draize tests. Dermatol. Sci. 2000 24:77-91.
Faller, C., Bracher, M., Dami, N. and Roguet, R., Predictive ability of reconstructed human epidermis equivalents for the assessment of skin irritation of cosmetics. Toxicol. In Vitro 2002 16:557-572.
Fedors, R. F., A method for estimating both the solubility parameters and molar volumes of liquids. Polymer Eng Sci. 1974 14:147-154.
Finnin, C. B. and Morgan, T. M., Transdermal penetration enhancers: Applications, limitations and potential. Journal of Pharmaceutical Sciences 1999 88:955-958.
Franz, T. J., The finite dose technique as a valid in vitro model for the percutaneous absorption in man. In Skin: Drug Application and Evaluation of Environmental Hazards (Current Problems in Dermatology, Vol. 7). Edited by Simon, G., Paster, Z., Klingberg, M., Kaye, M. and Karger, S., 1978 7:58-68.
Goates, C. Y. and Kuntson, K., Enhanced permeation of polar compounds through human epidennis. I. Permeability and membrane structural changes in the presence of short chain alcohols. Biochimica et Biophysica Acta 1994 1195:169-179.
Hansch, C. and Leo, A., Substituent constants for correlation analysis in chemistry and biology. Wiley and Sons, New York 1979.
Herzinger, T., Korting, H. and Maibach, H., Assessment of cutaneous and ocular irritancy: a decade of research on alternatives to animal experimentation. Fund. Appl. Toxicol. 1995 24:29-41.
Hoy, K. L., New values of the solubility parameters from vapor pressure data. J. Paint. Tech. 1970 42:76-78.
Jabs, A., Determination of secondary structure in proteins by Fourier transform infrared spectroscopy (FTIR). www.imb-iena.de/ImgLibDoc/ftir/IMAGE FTIR.html.
Kai, T., Mak, V. H. W., Potts, R. O. and Guy, R. H., Mechanism of percutaneous penetration by enhancement: effect of n-alkanols on the permeability barrier of hairless mouse skin. Journal of Controlled Release 1990 12:103-112.
Kanikkannan, N., Kandimalla, K., Lamba, S. S, and Singh, M., Structure-activity relationship of chemical penetration enhancers in transdermal drug delivery. Curr. Med. Chem. 2000 7:593-608.
Karande, P. and Mitragotri, S., High throughput screening of transdermal formulations. Pharmaceutical Research, 2002 19:655-660.
Kligman, A. and Christophel, E., Preparation of isolated sheets of human stratum corneum. Arch. Dermatol. 1963 88:702-705.
Klopman, G., Wang, S, and Balthasar, D. M., Estimation of aqueous solubility of organic molecules by the group contribution approach. Application to the study of biodegradation. J. Comp. Chem., 1992 32:474-482.
Kluge, T., Olejnik, J., Smilowitz, L. and Rothschild, K. J., Conformational changes in the core structure of bacteriorhodopsin. Biochemistry 1998 37:10279-10285.
Krimm, S, and Bandekar, J., Vibrational spectroscopy and conformation of peptides, polypeptides, and proteins. Adv. Protein Chem. 1986 38:181-364.
McGowan, J. C., A new approach for the calculation of hydrophile-lipophile balance values of surfactants. Tenside Surfactants 1990 27:229-230.
Mendelsohn, R., Mantsch, H. H., In Progress in Prorein-Lipid Interacrions. Watts, A., DePont J.J.H.H.M., Eds.; Elsevier: Amsterdam, 1986; Vol. 2, p. 103.
Mitragotri, S., Farrell, J., Tang, H., Terahara, T., Kost, J. and Langer, R., Determination of the threshold energy dose for ultra-sound-induced transdermal drug delivery. J. Control. Release 2000 63:41-52.
Moriguchi, I., Hirono, S., Liu, Q., Nakagome, I. and Matsushita, Y., Simple method of calculating octanol/water partition coefficient. Chem. Pharm. Bull. 1992 40:127-130.
Moriguchi, I., Hirono, S., Nakagome, L. and Hirano, H., Comparison of reliability of logP values for drugs calculated by several methods. Chem. Pharm. Bull. 1994 42, 976-978.
Newby, C., Barr, R., Greaves, M. and Mallet, A., Cytokine release and cytotoxicity in human keratinocytes and fibroblasts induced by phenols and sodium dodecyl sulfate. J. Invest. Dermatol. 2000 115:292-298.
Osborne, R. and Perkins, M. A., An approach for development of alternative test methods based on mechanisms of skin irritation. Food Chem. Toxicol. 1994 32:133-142.
Painter, P. C., Coleman, M. M., Koenig, J. L., The theory of vibrational spectroscopy and its application to polymeric materials. Wiley: New York, 1982.
Perkins, M. A., Osborne, R., Rana, F. R., Ghassemi, A. and Robinson, M. K., Comparison of in vitro and in vivo human skin responses to consumer products and ingredients with a range of irritancy potential. Toxicol. Sci. 1999 48:218-229.
Prausnitz, M. R., Mitragotri, S, and Langer, R., Current status and future potential of transdermal drug delivery. Nat. Rev. Drug Disc. 3:115-124, 2004.
Santus, C. G. and Baker, R. W., Transdermal enhancer patent literature. Journal of Controlled Release 1993 25:1-20.
Simon, G. and Maibach, H., The pig as an experimental animal model of percutaneous permeation in man: qualitative and quantitative observations—an overview. Skin Pharmacol. Physiol. 2000 13:229-234.
Van Krevelen, D. W., Properties of Polymers. 3rd Ed., Elsevier: Amsterdam, 1990.
Viswanadhan, V., Ghose, A., Revankar, G. and Robins, R., Atomic physicochemical parameters for three dimensional structure directed quantitative structure-activity relationships. 4. Additional parameters for hydrophobic and dispersive interactions and their application for an automated superposition of certain naturally occurring nucleoside antibiotics. J. Chem. Inf. Comput. Sci. 1989 29:163-172.
Walters, K. A., Penetration enhancers and their use in transdermal therapeutic systems. Transdermal Drug Delivery. Developmental Issues and Research Initiatives, ed. Hadgraft, J. and Guy R. H., 1989, New York: Marcel Dekker. 197-233.

1. A method for determining the irritation potential of a formulation comprising the following steps:
(a) providing a sample comprising stratum corneum;
(b) applying said formulation to said sample;
(c) measuring an infrared spectrum of said sample; and
(d) analyzing said infrared spectrum so as to determine the irritation potential of said formulation.

2. The method of claim 1 wherein said sample comprising stratum corneum is full thickness skin.

3. The method of claim 1 wherein said sample comprising stratum corneum consists essentially of stratum corneum.

4. The method of claim 1 wherein said infrared spectrum is measured using a Fourier transform infrared spectrometer.

5. The method of claim 1 wherein the resolution of said infrared spectrum is at least about 2 cm⁻¹.

6. The method of claim 1 wherein the irritation potential of said formulation is determined by analyzing the Amide I band of said infrared spectrum.

7. A method for determining the irritation potential of a formulation comprising the following steps:
(a) providing a sample comprising stratum corneum;
(b) measuring a first infrared spectrum of said sample;
(c) applying said formulation to said sample;
(d) rinsing said sample after a suitable incubation period;
(e) measuring a second infrared spectrum of said sample; and
(f) analyzing said first and second infrared spectra so as to determine the irritation potential of said formulation.

8. The method of claim 7 wherein said rinsing step utilizes deuterated solvents.

9. The method of claim 7 wherein the irritation potential of said formulation is determined by analyzing the changes in the α-helix contribution of stratum corneum proteins to the Amide I band of said first and second infrared spectra.

10. The method of claim 7 wherein said analyzing step comprises fitting said first and second infrared spectra to Gaussians.

11. A method for evaluating the ability of a molecule to enhance the transport of actives into or through skin comprising the following steps:
(a) providing a sample comprising stratum corneum;
(b) contacting a formulation comprising said molecule with said sample;
(c) collecting an infrared spectrum on said sample; and
(d) analyzing said infrared spectrum so as to evaluate the ability of said molecule to enhance the transport of actives into or through skin.

12. A method for determining the irritation potential of a molecule comprising the following steps:
(a) obtaining hydrogen bonding forces for said molecule;
(b) obtaining polar forces for said molecule; and
(b) utilizing the ratio of said hydrogen bonding forces to said polar forces to determine the irritation potential of said molecule.

13. A method for evaluating the potential of a molecule to enhance the permeability of skin comprising the following steps:
(a) obtaining logP data for said molecule; and
(b) utilizing said logP data to evaluate the potential of said molecule to enhance the permeability of skin.

14. A method for evaluating the potential of a molecule to enhance the permeability of skin comprising the following steps:
(a) obtaining the cohesive energy density data for said molecule;
(b) obtaining hydrogen bonding solubility parameters for said molecule; and
(c) utilizing said cohesive energy density data and said hydrogen bonding solubility parameters for said molecule to evaluate the potential of said molecule to enhance the permeability of skin.

15. An in silico method of identifying chemical penetration enhancers comprising the following steps:
(a) providing a plurality of molecules;
(b) obtaining a plurality of molecular descriptors for said plurality of molecules;
(c) utilizing said molecular descriptors to develop information on the potential of said molecules as chemical penetration enhancers; and
(d) analyzing said information to identify chemical penetration enhancers from said plurality of molecules.

16. A method for estimating the irritation potential of a formulation comprising the following steps;
(a) providing a sample comprising stratum corneum;
(b) applying said formulation to said sample;
(c) measuring the effects of said formulation on the structure of the proteins of said sample; and
(d) analyzing said measurements to estimate the irritation potential of said formulation.

17. A compound having the formula:

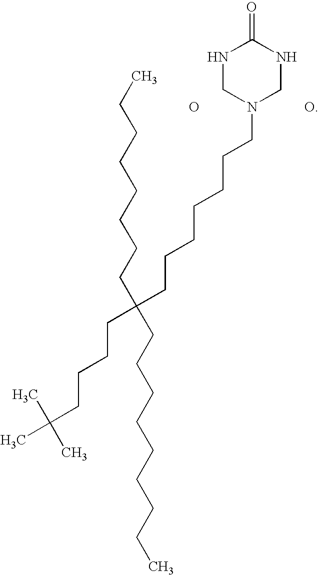

18. A compound having the formula:

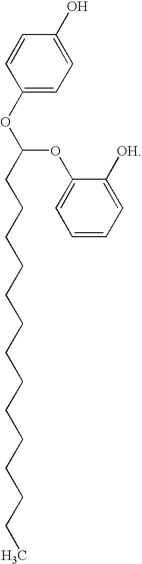

19. A compound having the formula:

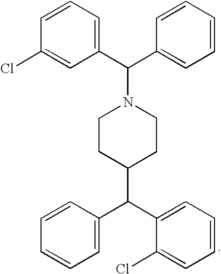

20. A compound having the formula:

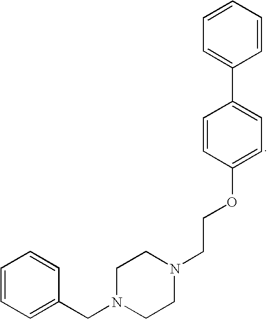

21. A compound having the formula:

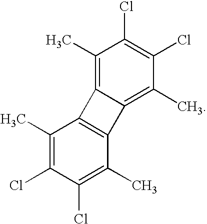

22. A method for administering an active component comprising applying to the skin of a human or animal a composition comprising an active component present in an amount effective to provide a desired effect and at least one compound selected from the group consisting of

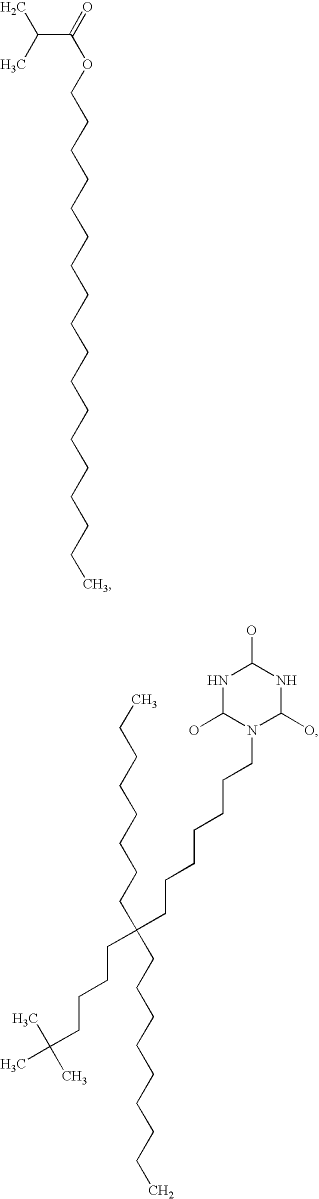

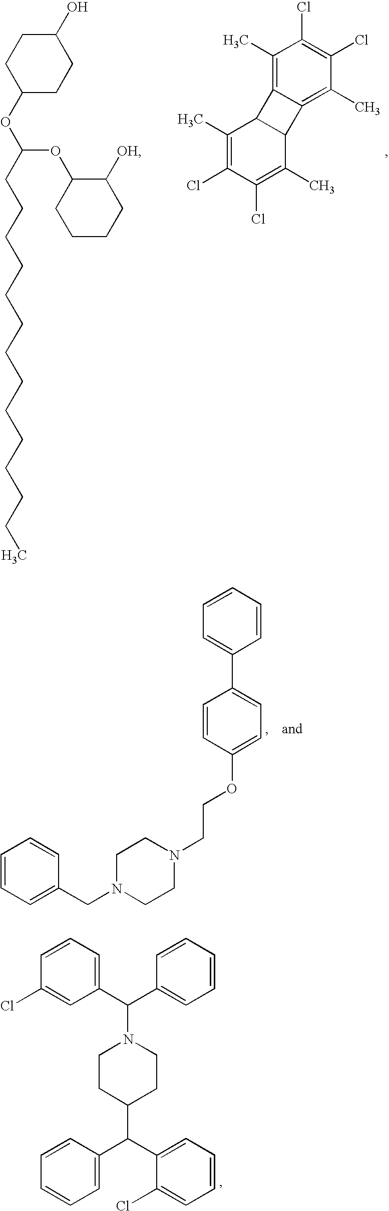

23. A method of administering an active component comprising applying to the skin of a human or animal a composition comprising an active component present in an amount to provide a desired effect and at least one compound with an ER/IP ratio of greater than 4.

24. The method of claim 23 wherein said at least one compound is stearyl methacrylate.
